(19) United States
(12) Patent Application Publication
(10) Pub. No.: US 2024/0050627 A1
(43) Pub. Date: Feb. 15, 2024

(54) CLASS OF BIOMATERIALS FOR PROMOTING LARGE BLOOD VESSEL GROWTH

(71) Applicant: Vanderbilt University, Nashville, TN (US)

(72) Inventors: Ethan S. Lippmann, Nashville, TN (US); Brian O'Grady, Nashville, TN (US); David C. Florian, Nashville, TN (US); Scott A. Guelcher, Nashville, TN (US)

(21) Appl. No.: 18/268,009

(22) PCT Filed: Dec. 17, 2021
(86) PCT No.: PCT/US21/64204

§ 371 Date: Jun. 16, 2023

Related U.S. Application Data
(60) Provisional application No. 63/127,808, filed on Dec. 18, 2020.
(60) Provisional application No. 63/244,905, filed on Sep. 16, 2021.
(60) Provisional application No. 63/276,358, filed on Nov. 5, 2021.
(60) Provisional application No. 63/187,844, filed on May 12, 2021.

(51) Int. Cl. A61L 27/50; A61L 27/52; A61L 27/54; A61L 27/22; A61L 27/18

(52) U.S. Cl. CPC A61L 27/507; A61L 27/52; A61L 27/54; A61L 27/222; A61L 27/18; A61L 2300/25

(57) ABSTRACT

Provided are biomaterials and methods useful for promoting large blood vessel growth in a subject. An example biomaterial includes a crosslinked hydrogel and a peptide chemically attached to the hydrogel wherein the peptide comprises an extracellular epitope of a cadherin protein. An example method includes administering to an area of the subject a therapeutically effective amount of the biomaterial, wherein the biomaterial provides artery growth, arteriole growth, a combination thereof in the area of administration.

### CROSS-REFERENCE TO RELATED APPLICATIONS

This application claims the benefit of and priority to U.S. Provisional Application No. 63/127,808, filed on Dec. 18, 2020; U.S. Provisional Application No. 63/187,844, filed on May 12, 2021; U.S. Provisional Application No. 63/244,905, filed on Sep. 16, 2021; and U.S. Provisional Application No. 63/276,358, filed on Nov. 5, 2021, the entire contents of each which are hereby incorporated by reference.

### STATEMENT OF GOVERNMENT INTEREST

This invention was made with government support under grants 1462866 and 1506717 awarded by the National Science Foundation. The government has certain rights in the invention.

### SEQUENCE LISTING

The instant application includes a Sequence Listing which has been submitted electronically in ASCII format and is hereby incorporated by reference in its entirety.

### INTRODUCTION

Healthy tissues require sufficient blood flow for oxygen and nutrient delivery. In aging and disease, arteries can become blocked, leading to ischemia in downstream tissue beds. Likewise, a significant number of transplanted organs are rejected due to ischemia-causing blood clots. Accordingly, it would be beneficial to have compositions and methods for treating ischemia.

### SUMMARY

In one aspect, the present disclosure provides a biomaterial including a crosslinked hydrogel and a peptide chemically attached to the hydrogel and including an extracellular epitope of a cadherin protein.

In another aspect, the present disclosure provides a method of promoting arteriogenesis for treating a disorder in a subject, the method including administering to an area of the subject in need thereof a therapeutically effective amount of a biomaterial, the biomaterial including a crosslinked hydrogel, and a peptide chemically attached to the hydrogel and including an extracellular epitope of a cadherin protein, wherein the biomaterial provides artery growth, arteriole growth, or a combination thereof in the area of administration.

In another aspect, the present disclosure provides a biomaterial including a crosslinked hydrogel comprising a multivalent polyethylene glycol (PEG) crosslinker; and a peptide chemically attached to the hydrogel and including an extracellular epitope of a cadherin protein.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 shows a schematic of biomaterials of the present disclosure relative to previous angiogenic biomaterials.

FIG. 2 shows a schematic illustration of methacrylated gelatin (GelMA) synthesis and N-cadherin peptide (e.g., N-Cadherin mimic also denoted as Cad) conjugation. The conventional method for synthesizing GelMA uses methacrylic anhydride to introduce a methacryloyl substitution group on the reactive primary amine group of amino acid residues. GelMA was then dissolved in TEAO buffer with the N-cadherin peptide for Michael-type addition to the reactive primary amine group of the amino acid.

FIG. 3 shows an assessment of biomaterial functionalization and physical properties of polymerized hydrogels. (A) nuclear magnetic resonance (NMR) spectra of gelatin, GelMA, GelMA-Cad, and GelMA-Scrambled peptide (Scram). Successful conjugation of methacrylic anhydride to the backbone of gelatin was assessed by peaks at 5.5 and 5.7 ppm, and N-cadherin/Scram peptide addition was assessed by the valine peak at 3.5 ppm. (B) Fourier-transform infrared (FTIR) spectra was used to confirm conjugation of the peptide to the backbone of GelMA due to decrease in the following relevant bands: 1000 cm⁻¹ (PO4 stretching) and 1250 cm⁻¹, 1540 cm⁻¹, and 1640 cm⁻¹ (NH bending). (C) atomic force microscopy (AFM) measurements of Young's modulus values for GelMA, GelMA-Cad, GelMA-Scram. Data are presented as mean±S.D. from 3 independently fabricated hydrogels, where three locations were sampled on each hydrogel as described in the methods.

FIG. 4 shows characterization of a hydrogel including a multivalent PEG crosslinker. (A) Gelation time can be influenced by lowering the pH of the gelation solution, the PEG solution, or both. (B) Rheometric studies showing that pH does not have a significant effect on the mechanical properties of the hydrogel.

FIG. 5 shows an assessment of patterned architectures in hydrogels fabricated from GelMA-Cad or Matrigel. Polydimethylsiloxane (PDMS) molds were filled with GelMA-Cad or Matrigel and crosslinked around a piece of silicone tubing, which was then manually removed. (A) GelMA-Cad hydrogel shows an intact channel that can be perfused. (B) The channel in the Matrigel hydrogel collapses after the tubing is removed.

FIG. 6 shows scanning electron microscopy (SEM) images of hydrogels fabricated from gelatin, GelMA, GelMA-Cad, and GelMA-Scram.

FIG. 7 shows a schematic of a process for preparing a hydrogel (gelatin) with attached peptide and a crosslinker (HPA).

FIG. 8 shows that the disclosed biomaterials promote arteriole growth. (A) In vitro vessel growth from brain tissue embedded in Cad but not control hydrogels. New vessels possess arteriole structure, grow millimeter distances, and deposit collagen. ZO-1 marks endothelial cells and α-SMA marks smooth muscle. (B) New arterioles morphologically resemble their tissue of origin. (C) Cad hydrogels were implanted in a mouse fat pad for two weeks, and the mouse heart was perfused with a fluorescent lipid before extraction of the hydrogel. The fluorescent images demonstrate that arterioles grew millimeter distances into the hydrogel and were properly connected to the host blood supply

FIG. 9 shows a schematic of biomaterials being implanted into the fat pads of mice to investigate large blood vessel formation.

FIG. 10 shows an image of a negative control Scram hydrogel fat pad sample. Fat pads and hydrogel are demarcated based on DAPI stain. The left image shows minimal cells in the hydrogel and numerous cells in the fat pad. The green label corresponds to ZO-1, a tight junction protein expressed in many different cell types—including endothelial cells. The right image shows a single small vessel growing into the hydrogel. However, there is no red signal associated with this vessel.

FIG. 11 shows an image of a disclosed Cad hydrogel fat pad sample. On the fat pad side, a perfused arteriole can be seen.

FIG. 12 shows an image of a disclosed Cad hydrogel fat pad sample. Dil image taken approximately 3 mm into the hydrogel. The vessels have arteriole size and architecture.

FIG. 13 shows an image of a Cad hydrogel implanted in a mouse fat pad over time. The left image shows a Cad hydrogel sample at 48 hours post-implant. It is hypothesized that the white appearance is due to cellular infiltration and remodeling of the hydrogel. The right image shows a Cad hydrogel sample at 2 weeks post-implant. The white appearance is more apparent at longer time intervals.

FIG. 14 shows an image of a Cad hydrogel with moderate stiffness and crosslinked with transglutaminase implanted for 2 weeks in a fat pad of a mouse. The left image is a side view. The right image is a top view. Lectin 568 labels endothelial cells red and DAPI labels cell nuclei blue.

FIG. 15 shows an image of a Scram hydrogel with moderate stiffness and crosslinked with transglutaminase implanted for 2 weeks in a fat pad of a mouse. The left image is a side view. The right image is a top view. Lectin 568 labels endothelial cells red and DAPI labels cell nuclei blue. Vessels from the surrounding tissue do not penetrate the gel.

FIG. 16 shows an image of a femoral artery injury model with an implanted Cad hydrogel. The image shows the placement of the sutures for the ligation model against known hallmarks. FA—femoral artery; FV—femoral vein; PCFA—proximal collateral femoral artery. The arrowheads outline the placement of the hydrogel. The hydrogel was delivered as a mixture of 5% modified gelatin and 5% 20 kDa PEG-SG.

FIG. 17 shows images of a hydrogel with 5% gelatin (w/v) crosslinked with a multivalent PEG crosslinker. (A) The image was taken 3 days post-implantation. The black box outlines the hydrogel. (B) This image shows the middle of the hydrogel at higher magnification.

FIG. 18 shows images of Cad and Scram hydrogels with 5% gelatin (w/v) crosslinked transglutaminase and implanted in a fat pad of a mouse. Mice were injected with microbubbles to visualize the perfused vessels. Arrowheads outline the hydrogel implant. A large blood vessel is noted in and around the Cad hydrogel at day 7. The visualized vessel is approximately 0.5 mm-1 mm in diameter.

FIG. 19 shows a series of representative images of mice that underwent a femoral artery ligation procedure and then administered a Cad hydrogel or control hydrogel with a scrambled peptide. The hydrogel was 5% gelatin crosslinked with a multivalent PEG crosslinker. The left side denoted Cad corresponds to a hydrogel including an extracellular epitope of a cadherin protein. The right side denoted Scram corresponds to a control hydrogel with a scrambled peptide. The days post procedure/implant is noted in white and indicates day 1, 7, 10, and 14 post-procedure/implant.

FIG. 20 shows a plot of recovery from femoral artery ligation (e.g., ischemia) as measured by Tarlov score. The light blue bars correspond to GelCad (n=3). The light green bars correspond to the control, GelScram (n=3 for each time point but day 14, where n=2). Tarlov score is as follows: 0—no movement; 1—barely perceptible movement, no weight bearing; 2—frequent and vigorous movement, no weight bearing; 3—support weight, may take 1 or 2 steps; 4—walks with only mild deficit; 5—normal but slow walking; and 6—full and fast walking.

FIG. 21 shows a plot of recovery from femoral artery ligation as measured by Ischemia score. The light blue bars correspond to GelCad (n=3). The light green bars correspond to the control, GelScram (n=3 for each time point but day 14, where n=2). Ischemia score is as follows: 0—auto amputation>half lower limb: 1—gangrenous tissue>half foot: 2—gangrenous tissue<half foot, with lower limb muscle necrosis; 3—gangrenous tissue<half foot, without lower limb muscle necrosis; 4—pale foot or gait abnormalities; and 5—normal.

FIG. 22 shows a plot of recovery from femoral artery ligation as measured by Modified Ischemia score. The light blue bars correspond to GelCad (n=3). The light green bars correspond to the control, GelScram (n=3 for each time point but day 14, where n=2). Modified ischemia score is as follows: 0—autoamputation of leg; 1—leg necrosis; 2—foot necrosis; 3—two or more toe discoloration; 4—one toe discoloration; 5—two or more nail discoloration; 6—one nail discoloration; and 7—no necrosis.

FIG. 23 shows a plot of recovery from femoral artery ligation as measured by perfusion index. The light blue bars correspond to GelCad (n=3). The light green bars correspond to the control, GelScram (n=3 for each time point but day 14, where n=2).

FIG. 24 shows a representative histology image following femoral artery ligation of a mouse that was implanted with GelCad. Hydrogel remnants appear to be present subcutaneously and are surrounded and infiltrated by abundant numbers of mixed immune cells, primarily neutrophils and macrophages, which are then surrounded by numerous, tightly packed plasma cells and fewer lymphocytes (all outlined in a dotted line). Intermixed within these immune cells and surrounding the biomaterial are plump fibroblasts along with peripheral neovascularization. No significant pathology is noted within the bone, marrow, and skeletal muscle.

FIG. 25 shows a representative histology image following femoral artery ligation of a mouse that was implanted with GelScram. Subcutaneously there are abundant numbers of mixed immune cells, primarily neutrophils and macrophages, which are then surrounded by numerous, tightly packed plasma cells and fewer lymphocytes. This is around empty space which could be where the hydrogel was and/or suture material. Intermixed within this inflammation and surrounding the biomaterial are plump fibroblasts along with peripheral neovascularization. In this section, there is marked, extensive myofiber shrinkage and loss (degeneration) and hyperesosinophilia (necrosis) along with myofiber regeneration. There is a nerve also in section (likely femoral nerve) which is vacuolated indicative of axonal degeneration. There is also noted evidence of foreign material which could be the sutures (see arrows).

FIG. 26 shows representative images from mouse brain tissue embedded in different hydrogel formulations. In gelatin and GelScram, few of the sprouted vessels have smooth muscle cells marked by α-SMA. In GelCad, smooth muscle cells are detected within 24 hours and mature into ring-like structures that mimic native arteriole architecture by 7 days.

FIG. 27 shows an image of vessels sprouted from mouse brain tissue into the GelCad hydrogel are actively proliferating, as determined by Ki-67 staining.

FIG. 28 shows images of large blood vessel growth from disclosed biomaterials. FIG. 28A shows α-SMA+/lectin+arterioles sprouting from embedded human brain tissue down into lectin+/α-SMA-capillaries. FIG. 28B shows an arteriole immunostained for α-SMA and ZO-1, revealing concentric, ring-like smooth muscle coverage. FIG. 28C shows a cross-section of an arteriole, where a lumen has formed with ZO-1+ endothelial cells on the inside and α-SMA+smooth muscle cells on the outside.

### DETAILED DESCRIPTION

Occlusion of large arteries reduces blood flow and oxygen delivery to downstream tissues. Depending on the location and severity of the blockage, resultant tissue ischemia can lead to debilitating pain, poor wound healing, myocardial infarction, stroke, limb amputation, and organ failure. A substantial amount of research has been devoted towards inducing collateral arteriole growth (e.g., arteriogenesis) for restoration of blood flow, but to date, all clinical trials utilizing systemic delivery of small molecules, growth factors, cells either alone or as part of a scaffold system have failed to produce any therapeutic benefit as related to the generation of arteriole growth.

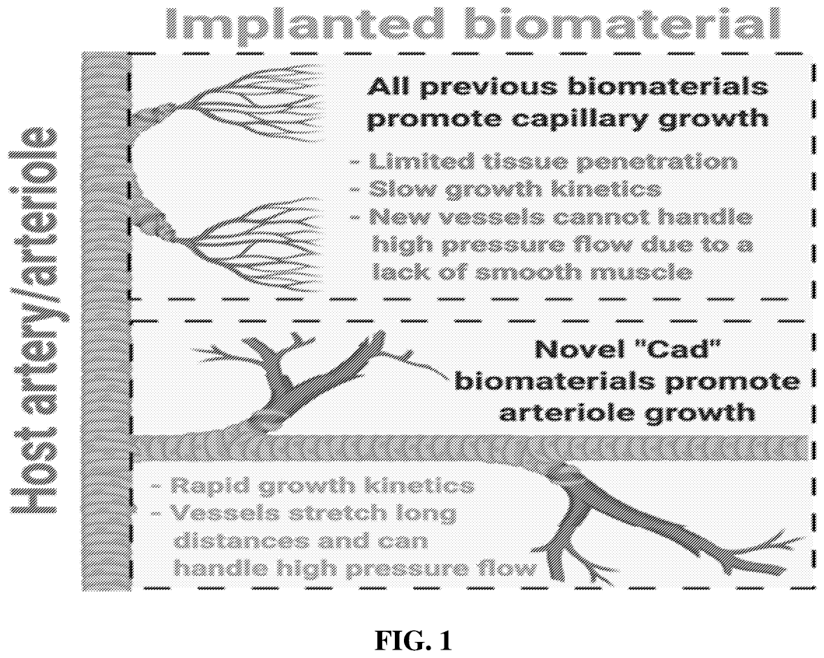

Hydrogels represent a potential useful strategy for inducing arteriogenesis since they can be locally implanted or injected and engineered to promote desired bioactivity. However, all previous hydrogels have been designed to induce growth of capillaries rather than arterioles (i.e. angiogenesis rather than arteriogenesis) (FIG. 1). Unlike arteries and arteriole vessels, capillaries are the smallest and most fragile vessels in the body. Capillary growth is very slow, which limits tissue penetration of newly formed vessels. In addition, since capillaries lack smooth muscle, they cannot handle high pressure outflows from arteries. Thus, biomaterials that can promote capillary growth, but not larger vessel growth such as arteries, are limited in their application.

The present disclosure relates to a new class of biomaterials that promote large blood vessel growth, such as arteries and arterioles. The disclosed biomaterials include a crosslinked hydrogel (e.g., gelatin) that has chemically attached peptides that include a conserved extracellular epitope from Type 1 cadherins, including N-cadherin. The present disclosure found that these biomaterials are potent inducers of arteriole and artery growth both in vitro and in vivo. It was found that primary mouse and human tissues, upon embedding in the hydrogels, rapidly sprout large blood vessels in the biomaterials, where these vessels are millimeters in length and exhibit biomimetic organization of smooth muscle cell rings lining an inner endothelial cell core. The present disclosure found that new artery growth was achieved in vitro from brain and bone marrow—suggesting that large blood vessel growth was independent from tissue type. Furthermore, the present disclosure found that perfusable arteries rapidly grow into the biomaterials when implanted in vivo where the biomaterial was implanted in both fat pads and hindlimbs of mice—suggesting that growth is independent of location. Accordingly, the disclosed biomaterials and methods thereof provide a beneficial technology for the treatment of ischemia and related disorders.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%/6” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkyl” as used herein, means a straight or branched chain saturated hydrocarbon. Representative examples of alkyl include, but are not limited to, methyl, ethyl, n-propyl, isopropyl, n-butyl, sec-butyl, isobutyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “crosslinker” as used herein refers to a molecule or a functional group capable of linking one polymer to another polymer, or one part of a polymer to another part of the polymer, via formation of one or more chemical bonds between the two polymers or the two parts of the polymer.

The term “chemically bonding” or “chemically attaching” as used herein refers to forming a chemical bond between two substances. The chemical bond may be an ionic bond, a covalent bond, dipole-dipole interaction, or hydrogen bond.

The terms “effective amount” or “therapeutically effective amount,” as used herein, refer to a sufficient amount of a biomaterial being administered which will relieve to some extent one or more of the symptoms of the disease or disorder being treated. The result can be reduction and/or alleviation of the signs, symptoms, or causes of a disease, or any other desired alteration of a biological system. For example, an “effective amount” for therapeutic uses is the amount of the biomaterial as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration, the type or extent of supplemental therapy used, ongoing disease process and type of treatment desired (e.g., aggressive vs. conventional treatment).

The term “ischemia,” as used herein, refers to any decrease or stoppage in the blood supply to any cell, tissue, organ, or body part caused by any constriction, damage, or obstruction of the vasculature. Ischemia sometimes results from vasoconstriction or thrombosis or embolism. Ischemia can lead to direct ischemic injury, tissue damage due to cell death caused by reduced supply of oxygen (hypoxia, anoxia), glucose, and nutrients. “Hypoxia” or a “hypoxic condition” intends a condition under which a cell, organ or tissue receives an inadequate supply of oxygen. “Anoxia” refers to a virtually complete absence of oxygen in the organ or tissue, which, if prolonged, may result in death of the cell, organ or tissue.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. All amino acid residue sequences are represented herein by formulae with left and right orientation in the conventional direction of amino-terminus to carboxy-terminus.

“Substantially identical” means that a first and second amino acid sequences are at least 60%, 65%, 70%, 75%, 80%, 85%, 90%, 95%, 96%, 97%, 98%, or 99% identical over a region of 10, 20, 30, 40, 50, 60, 70, 80, 90, or even 100 amino acids.

A “variant” refers to a peptide or polypeptide that differs in amino acid sequence by the insertion, deletion, or conservative substitution of amino acids, but retain at least one biological activity. Representative examples of “biological activity” include, for example, the ability to promote cell adhesion, to be bound by a specific antibody or polypeptide, or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids. See Kyte et al., J. Mol. Biol. 1982, 157, 105-132. The hydropathic index of an amino acid is based on a consideration of its hydrophobicity and charge. It is known in the art that amino acids of similar hydropathic indexes can be substituted and still retain protein function. In one aspect, amino acids having hydropathic indices of 2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S. Pat. No. 4,554,101, which is fully incorporated herein by reference. Substitution of amino acids having similar hydrophilicity values can result in polypeptides retaining biological activity, for example cell adhesion, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof. In some embodiments, variants include homologues. Homologues may be polypeptides or genes inherited in two species by a common ancestor.

The term “conservative change” refers to a change made to an amino acid sequence without altering activity. These changes are termed conservative substitutions or mutations; that is, an amino acid belonging to a grouping of amino acids having a particular size or characteristic can be substituted for another amino acid. Substitutes for an amino acid sequence may be selected from other members of the class to which the amino acid belongs. For example, the nonpolar (hydrophobic) amino acids include alanine, leucine, isoleucine, valine, proline, phenylalanine, tryptophan, and tyrosine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Such alterations are not expected to substantially affect apparent molecular weight as determined by polyacrylamide gel electrophoresis or isoelectric point. Exemplary conservative substitutions include, but are not limited to, Lys for Arg and vice versa to maintain a positive charge; Glu for Asp and vice versa to maintain a negative charge; Ser for Thr so that a free —OH is maintained; and Gln for Asn to maintain a free NH₂. Moreover, point mutations, deletions, and insertions of the polypeptide sequences or corresponding nucleic acid sequences may in some cases be made without a loss of function of the polypeptide or nucleic acid fragment.

The terms “treatment” or “treating,” as used herein, refer to protection of a subject from a disease or disorder, means preventing, suppressing, repressing, ameliorating, or completely eliminating the disease or disorder. Preventing the disease or disorder involves administering a biomaterial of the present disclosure to a subject prior to onset of the disease. Suppressing the disease involves administering a biomaterial of the present disclosure to a subject after induction of the disease or disorder but before its clinical appearance. Repressing or ameliorating the disease or disorder involves administering a biomaterial of the present disclosure to a subject after clinical appearance of the disease or disorder.

As it relates to ischemia, “treatment” or “treating” can include: (a) preventing the ischemia from occurring in a subject which may be predisposed to the ischemia but has not yet been diagnosed as having it; (b) inhibiting the ischemia or preventing further ischemic tissue damage, i.e., arresting its development; (c) relieving the ischemia, e.g., causing revascularization of the tissue, e.g., to completely or partially eliminate symptoms of the ischemia; and/or (d) restoring the individual to a pre-disease state, e.g., complete revascularization of the previously ischemic tissue. “Treatment” may not indicate, or require, complete eradication or cure of the ischemia, or associated symptoms thereof. In particular methods of the disclosure, “treatment” or “treating” provides improved blood flow to an ischemic tissue, improved oxygenation of an ischemic tissue, improved vascularization of an ischemic tissue, and/or improved survival of ischemic tissue.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. BIOMATERIALS

In one aspect, provided is a biomaterial comprising a crosslinked hydrogel and a peptide chemically attached to the hydrogel, wherein the peptide comprises an extracellular epitope of a cadherin protein. In some embodiments, the biomaterial is a crosslinked hydrogel including a peptide chemically attached to the hydrogel and including an extracellular epitope of a cadherin protein.

The disclosed biomaterials may function as an extracellular matrix (ECM) material useful in tissue culture and the treatment of disorders such as ischemia. Suitable hydrogels, peptides, and crosslinkers are selected such that the resulting biomaterials as disclosed herein may (1) facilitate large blood vessel growth (e.g., arteriogenesis), (2) exhibit ideal mechanical properties to promote large blood vessel growth, and/or (3) be relatively easy to synthesize, low cost, and therefore widely accessible.

The biomaterial may include further molecules that can aid in the growth of large blood vessels. For example, the biomaterial may include a plurality of cells, proteins—such as growth factors, genetic material—such as siRNA, or a combination thereof. Cells can include, but are not limited to, vascular cells, connective tissue cells, stem cells, and tissue-specific cells derived from stem cells. In some embodiments, the biomaterial does not include any other molecule. In some embodiments, the biomaterial does not include any cells.

A. Hydrogels

The hydrogel may include a polymeric material. For example, the hydrogel may be a polymeric material having a network of hydrophilic polymers. In some embodiments, the polymeric material is a hydrophilic polymer. The polymeric material may be natural or synthetic polymers, and may include known polymers used for tissue engineering, cell culture, biosensors, implants, etc. Suitable hydrogels include hydrogels comprising one or more of hyaluronic acid, polyethylene glycol, polypropylene glycol, polyethylene oxide, polypropylene oxide, polyglutamate, polylysine, polysialic acid, polyvinyl alcohol, polyacrylate, polymethacrylate, polyacrylamide, polymethacrylamide, polyvinyl pyrrolidone, polyoxazoline, polyiminocarbonate, polyamino acid, hydrophilic polyester, polyamide, polyurethane, polyurea, dextran, agarose, xylan, mannan, carrageenan, alginate, gelatin, collagen, albumin, cellulose, methylcellulose, ethylcellulose, hydroxypropylmethylcellulose, hydroxyethyl starch, chitosan, nucleic acids, derivatives thereof, co-polymers thereof, or combinations thereof. Examples of natural hydrogels include those derived from animal tissues, such as gelatin. In some embodiments, the polymeric material includes gelatin. For example, the hydrogel may be gelatin, or may include a variant or derivative of gelatin. In some embodiments, the hydrogel includes gelatin and one or more other components, such as a hydrophilic polymeric component (e.g. PEG), a hyaluronic acid, or chitosan. In some embodiments, the hydrogel comprises gelatin, such as animal skin gelatin. In particular embodiments, the hydrogel comprises porcine skin gelatin.

The hydrogel may include the polymeric material at varying amounts. For example, the biomaterial may include the polymeric material at about 1% to about 20% by weight of the hydrogel, such as about 2% to about 18% by weight of the hydrogel, about 3% to about 15% by weight of the hydrogel, or about 5% to about 10% by weight of the hydrogel. The amount of the polymeric material can be modulated to manipulate properties of the hydrogel and biomaterial thereof.

B. Peptides

The peptide may comprise a flanking sequence at the N-terminal end, the C-terminal end, or both the N- and C-terminal ends of the HAV sequence. The peptide may be chemically attached to the hydrogel at the N-terminal end or the C-terminal end. For example, the peptide may be attached to the hydrogel through a residue at the C-terminal end. The amino acid through which the peptide is attached to the hydrogel may be a polar amino acid, such as cysteine (Cys) or glutamic acid (Glu). In some embodiments, the peptide is attached to the hydrogel through a C-terminal Cys or C-terminal Glu. In some embodiments, the peptide is attached to the hydrogel at the C-terminal end, and the N-terminal end of the peptide include a known tag or modification, such as an acetyl group (Ac). In some embodiments, the peptide is attached to the hydrogel via a C-terminal Cys or a C-terminal Glu, and the N-terminal end of the peptide is acetylated.

In some embodiments, the peptide is 5 to 30 amino acids in length. The peptide may include at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, or at least 29 amino acids. The peptide may include less than 30, less than 29, less than 28, less than 27, less than 26, less than 25, less than 24, less than 23, less than 22, less than 21, less than 20, less than 19, less than 18, less than 17, less than 16, less than 15, less than 14, less than 13, less than 12, less than 11, or less than 10 amino acids. The peptide may be 5 to 25 amino acids in length, 8 to 25 amino acids in length, 8 to 15 amino acids in length, or 8 to 12 amino acids in length. In some embodiments, the peptide is 8 to 12 amino acids in length. In particular embodiments, the peptide is 9 or 10 amino acids in length.

The peptide may be included in the hydrogel at varying densities. The method of measuring the degree of functionalization with the peptide can take into account different properties of the peptide and the hydrogel. For example, the percentage of a valine NMR peak (which is present on the peptide but not on a gelatin hydrogel) relative to a phenylalanine NMR peak (which is on a gelatin hydrogel but not the peptide) can be used to measure the degree of functionalization. The hydrogel may include the peptide at a degree of functionalization of about 10% to about 70% as measured by the amount of valine of the peptide relative to the amount of phenylalanine of the hydrogel per NMR, such as about 20% to about 65% as measured by the amount of valine of the peptide relative to the amount of phenylalanine of the hydrogel per NMR, about 30% to about 60% as measured by the amount of valine of the peptide relative to the amount of phenylalanine of the hydrogel per NMR, or about 35% to about 70% as measured by the amount of valine of the peptide relative to the amount of phenylalanine of the hydrogel per NMR.

In some embodiments, the peptide comprises an extracellular epitope of a cadherin protein, or a variant thereof. The term “cadherin” refers to a family of cell surface proteins, which may participate in Ca²⁺-dependent cell adhesion. Some subfamilies of cadherins are considered classical cadherins, which have multiple extracellular domains, a transmembrane domain, and a cytoplasmic domain. Examples of known cadherins include N-cadherin, E-cadherin, and P-cadherin. Sequences of cadherin proteins and variants thereof include those described in Kister et al. (Protein Sci., 2001, 10(9): 1801-1810), Renaud-Young et al. (J. Biol. Chem., 2002, 277(42), 39609-39616), Williams et al. (J Biol Chem., 2002, 277(6),4361-4367), and Williams et al. (J Biol Chem., 2000, 275(6), 4007-4012), the entire contents of each which are incorporated herein by reference.

In some embodiments, the peptide comprises an extracellular epitope of a cadherin protein with one or more conservative changes. In some embodiments, the peptide comprises a sequence that is substantially identical to an extracellular epitope of a cadherin protein. For example, the peptide may comprise a sequence that is at least 85%, at least 90%, at least 91%, at least 92%, at least 93%, at least 94%, at least 95%, at least 96%, at least 97%, at least 98%, or at least 99% identical to an extracellular epitope of a cadherin protein. In some cases a determination of the percent identity of a peptide to a sequence set forth herein (e.g., a Cadherin protein sequence) may be required. In such cases, the percent identity is measured in terms of the number of residues of the peptide, or a portion of the peptide. A peptide of, e.g., 90% identity, may also be a portion of a larger peptide. Embodiments include such peptides that have the indicated identity and/or conservative substitution of a cadherin sequence set forth herein, with said polypeptides exhibiting specific cell adhesion activities.

In some embodiments, the HAV sequence is at the N-terminal end of the peptide. In some embodiments, the peptide further comprises a Asp-Ile-Gly-Gly (DIGG) sequence, a Asp-Ile-Asn-Gly (DING) sequence, a Ser-Ser-Asn-Gly (SSNG) sequence, or a Ser-Glu-Asn-Gly (SENG) sequence. The DIGG, DING, SSNG, or SENG sequence may be to the C-terminal of the HAV sequence. For example, the DIGG, DING, SSNG, or SENG sequence may be attached to the C-terminal end of the HAV sequence. In some embodiments, the peptide comprises the sequence of HAVD1 at the N-terminal end of the peptide.

In some embodiments, the peptide comprises SEQ ID NO: 1 (HAVDIGGGC), SEQ ID NO: 2 (HAVDIGGGCE), a variant of SEQ ID NO: 1 or SEQ ID NO: 2, or a combination thereof. In some embodiments, the peptide consists of SEQ ID NO: 1, SEQ ID NO: 2, or a variant thereof. In some embodiments, the peptide includes at least one additional amino acid at the C-terminal end, at the N-terminal end, or at both the C-terminal and N-terminal ends, of the amino acid sequence of SEQ ID NO: 1 or SEQ ID NO: 2. In some embodiments, the peptide includes sequence tags or modifications as known in the art to the C-terminal end, the N-terminal end, or both the C-terminal and N-terminal ends of SEQ ID NO: 1 or SEQ ID NO: 2. In some embodiments, the peptide includes an acetyl group (Ac) at the N-terminal end of the amino acid sequence of SEQ ID NO: 1 (Ac-HAVDIGGGC) or SEQ ID NO: 2 (Ac-HAVDIGGGCE).

C. Crosslinking

The hydrogel may be crosslinked by various known methods. In some embodiments, the hydrogel is crosslinked by enzymatic crosslinking, thermal crosslinking, a crosslinker, or a combination thereof. In some embodiments, the hydrogel is crosslinked through non-covalent interactions, such as those seen with guest-host interactions. In some embodiments, the hydrogel is crosslinked via a crosslinker comprising an acrylate, a phenol group, a multivalent PEG, or a combination thereof. Accordingly, the hydrogel may include a crosslinker.

The crosslinker may be biodegradable. For example, the crosslinker may have ester linkages that allow for hydrolysis. The crosslinker may have varying rates of biodegradability based on, e.g., the amount of ester linkages, molecular weight, and other structural features of the crosslinker.

In some embodiments, the hydrogel includes proteins or polypeptides, which may be crosslinked by a suitable enzyme catalyzing the formation of a chemical bond between proteins and polypeptides. For example, the crosslinking may be catalyzed by a transglutaminase, such as a microbial transglutaminase, which catalyzes the formation of isopeptide bonds between proteins. Suitable techniques for enzymatic crosslinking of protein-containing hydrogels include those described in O'Grady et al. (SLAS Technology, 2018, 23(6). 592-598), which is incorporated herein by reference in its entirety.

In some embodiments, the hydrogel may be crosslinked with a thermal free radical initiator. Suitable thermal initiators include azo-based radical initiators. Examples of this class of initiators include 2,2′-Azobis[2-(2-imidazolin-2-yl)propane]dihydrochloride (VA-044) and 2,2′-Azobis[2-methyl-N-(2-hydroxyethyl)propionamide] (VA-086). Suitable techniques for thermally crosslinking a hydrogels include those described in Zhen et al. (Brain Struct. Funct. 2016, 221(4), 2375-2383), which is incorporated herein by reference in its entirety.

In some embodiments, the hydrogels may be crosslinked by any suitable crosslinker that does not interfere with the function of the biomaterial to facilitate cell growth. The crosslinker may have at least one function group for attachment to the hydrogel and at least one crosslinkable group. In general, attachment of the crosslinker to the hydrogel provides a crosslinkable hydrogel, which may be crosslinked under suitable conditions. Various crosslinkers for making a crosslinkable hydrogel are known in the art. Suitable crosslinkers may include, for example, an UV-light activated crosslinker, a redox-activated crosslinker, a thermal polymerization initiator, or a combination thereof. Suitable crosslinkers may include those described in U.S. Pat. Nos. 5,686,504, 8,287,906, and WO 2019/055656, the entire contents of each which are incorporated herein by reference.

Suitable UV-light activated crosslinkers include those having a vinyl group (—CH═CH₂). The vinyl group may be optionally substituted, for example, with an alkyl group. Examples of UV-light activated crosslinkers include alkyl acrylic acids, such as methacrylic acid, ethyl acrylate, n-propyl acrylate, isopropyl acrylate, n-butyl acrylate, isobutyl acrylate, n-amyl acrylate, iso-amyl acrylate, n-hexyl acrylate, isohexyl acrylate, cyclohexyl acrylate, isooctyl acrylate, 2-ethylhexy acrylate, decyl acrylate, lauryl acrylate, stearyl acrylate, or isobornyl acrylate. In particular embodiments, the hydrogel is crosslinked by methacrylic acid (HOOC—C(CH₂)═CH₂).

Suitable redox-activated crosslinkers include those having a phenol group (—C₄H₄OH). Examples of the crosslinkers having a phenol group include tyrosine (Tyr) and 3-(4-hydroxyphenyl)propionic acid (HPA). In some embodiments, the hydrogel with attached redox-activated crosslinkers is crosslinked by an oxidation reaction. In particular embodiments, a hydrogel with attached HPA is crosslinked by an oxidative coupling of HPA moieties catalyzed by hydrogen peroxide (H₂O₂) and horseradish peroxidase (HRP). Suitable techniques for crosslinking a hydrogel using redox-activated crosslinkers include those described in Wang et al. (Biomaterials, 2010, 31(6), 1148-1157), which is incorporated herein by reference in its entirety.

In some embodiments, the crosslinker is a multivalent PEG. The multivalent PEG crosslinker may be biodegradable. For example, the multivalent PEG crosslinker may include an ester linkage(s). The multivalent PEG crosslinker may have 1, 2, 3, 4, 5, or 6 functional groups that can each, individually form a chemical bond with a functional group of the hydrogel. For example, the multivalent PEG crosslinker may have 1 to 6 functional groups that can form a chemical bond with an amine of the hydrogel. In some embodiments, the multivalent PEG crosslinker includes an acrylate group, a succinimidyl group, or a combination thereof. In some embodiments, the multivalent PEG crosslinker is a multivalent succinimidyl glutarate. In some embodiments, the multivalent PEG crosslinker has 4 functional groups that can form a chemical bond with functional groups of the hydrogel.

The multivalent PEG crosslinker may have a varying molecular weight, which can aid in modulating degradation rates of the hydrogel. The multivalent PEG crosslinker may have a number average molecular weight of about 1 kDa to about 40 kDa, such as about 1.5 kDa to about 30 kDa, about 2 kDa to about 25 kDa, or about 2 kDa to about 20 kDa. Molecular weight can be measured by any technique known in the art including, but not limited to, gel permeation chromatography, light scattering, and viscosity measurements.

In some embodiments, the hydrogel is porcine skin gelatin, the peptide is a SEQ ID NO. 1, and the hydrogel is crosslinked by methacrylic acid. The resulting biomaterial may be referred to as “GelMA-Cad,” which includes methacrylated gelatin (GelMA, capable of being photopatterned) conjugated with a peptide from an extracellular epitope of N-cadherin.

In some embodiments, the hydrogel is porcine skin gelatin, the peptide is a SEQ ID NO. 2, and the hydrogel is crosslinked by 3-(4-hydroxyphenyl)propionic acid.

In some embodiments, the biomaterial includes a crosslinked hydrogel comprising a multivalent PEG crosslinker; and a peptide chemically attached to the hydrogel and comprising an extracellular epitope of a cadherin protein.

D. Preparation

In another aspect, provided is a method of preparing a biomaterial, comprising: chemically attaching a peptide comprising a histidine-alanine-valine (HAV) sequence to a hydrogel; and crosslinking the hydrogel having the attached peptide.

The hydrogel, peptide, and crosslinking processes are as described herein. In some embodiments, the hydrogel used for preparing the biomaterial comprises gelatin. In particular embodiments, the hydrogel used for preparing the biomaterial comprises porcine skin gelatin.

In some embodiments, the peptide used for preparing the biomaterial comprises SEQ ID NO: 1, SEQ ID NO: 2, a variant thereof, or a combination thereof.

In some embodiments, the crosslinking process comprises enzymatic crosslinking, thermal crosslinking, chemically attaching a crosslinker to the hydrogel, or a combination thereof. Suitable regents and techniques for enzymatic crosslinking and thermal crosslinking processes, and suitable crosslinkers are as described herein. In some embodiments, the crosslinking comprises chemically attaching a crosslinker to the hydrogel; and crosslinking the hydrogel having the attached peptide and the attached crosslinker.

In some embodiments, a method of preparing a biomaterial is provided, which comprises: chemically attaching a peptide comprising a histidine-alanine-valine (HAV) sequence to a hydrogel; chemically attaching a crosslinker to the hydrogel; and crosslinking the hydrogel having the attached peptide and the attached crosslinker.

In some embodiments, the peptide is chemically attached to the hydrogel prior to attaching the crosslinker to the hydrogel. In some embodiments, the crosslinker is chemically attached to the hydrogel prior to attaching the peptide. When the crosslinker is attached to the hydrogel prior to the attachment of the peptide, the peptide may be attached to the hydrogel at a position not occupied by the crosslinker, and/or to a crosslinker attached to the hydrogel.

In some embodiments, the crosslinker used for preparing the biomaterial includes a UV-light activated crosslinker, a redox-activated crosslinker, a chemical crosslinker or a combination thereof. In some embodiments, the crosslinker used for preparing the biomaterial has an optionally substituted vinyl group, an optionally substituted phenol group, a succinimidyl group or a combination thereof.

In some embodiments, the crosslinker has a —C(CH₃)═CH₂ group. In particular embodiments, the crosslinker is methacrylic acid. In these embodiments, the crosslinking step may be initiated by UV light (such as a 25 mW/cm² UV light exposure) in the presence of a photoinitiator. Examples of photoinitiators include lithium phenyl-2,4,6-trimethylbenzoylphosphinate (LAP). In some embodiments, the crosslinker is methacrylic acid, and the crosslinking step may initiate by exposing the hydrogel having the attached peptide and the attached crosslinker to photoinitiator LAP and UV light.

In some embodiments, the crosslinker is a UV-light activated crosslinker (e.g., one having a —C(CH)═CH₂ group), and the crosslinker is chemically attached to the hydrogel prior to the attachment of the peptide to the hydrogel. The subsequently attached peptide may be chemically attached to the hydrogel at a position not occupied by the crosslinker, and/or to a crosslinker attached to the hydrogel. In some embodiments, a method of preparing a biomaterial is provided, which comprises: chemically attaching a UV-light activated crosslinker to a hydrogel to form a crosslinkable hydrogel; chemically attaching a peptide comprising a histidine-alanine-valine (HAV) sequence to the crosslinkable hydrogel; and exposing the resulting hydrogel to UV light, thereby causing the hydrogel to crosslink.

In some embodiments, the crosslinker is methacrylic acid, which is chemically attached to the hydrogel (such as gelatin) prior to the attachment of the peptide to the hydrogel. The subsequently attached peptide may be chemically attached to the hydrogel at a position not occupied by methacrylic acid, and/or to a methacrylic acid moiety attached to the hydrogel. In particular embodiments, a method of preparing a biomaterial is provided, which comprises: chemically attaching methacrylic acid to a hydrogel to form a methacrylated hydrogel; chemically attaching a peptide comprising a histidine-alanine-valine (HAV) sequence to the methacrylated hydrogel; and exposing the resulting hydrogel to UV light, thereby causing the hydrogel to crosslink.

In some embodiments, the crosslinker is a redox-activated crosslinker. In some embodiments, the crosslinker is a redox-activated crosslinker having a phenol group, such as 3-(4-hydroxyphenyl)propionic acid. In these embodiments, one crosslinker may form a covalent bond with another crosslinker under oxidative conditions, for example, horseradish peroxidase (HRP) and H₂O₂. In some embodiments, a method of preparing a biomaterial is provided, which comprises: chemically attaching a peptide comprising a histidine-alanine-valine (HAV) sequence to form a functionalized hydrogel; chemically attaching a redox-activated crosslinker to the functionalized hydrogel; and subjecting the resulting hydrogel to an oxidation reaction, thereby causing the hydrogel to crosslink.

In particular embodiments, the crosslinker is 3-(4-hydroxyphenyl)propionic acid. A method of preparing a biomaterial is provided, which comprises: chemically attaching a peptide comprising a histidine-alanine-valine (HAV) sequence to form a functionalized hydrogel; chemically attaching 3-(4-hydroxyphenyl)propionic acid to the functionalized hydrogel; and subjecting the resulting hydrogel to an oxidation reaction, thereby causing the hydrogel to crosslink.

In particular embodiments, the crosslinker is a multivalent PEG succinimidyl glutarate. A method of preparing a biomaterial is provided, which comprises: chemically attaching a peptide comprising a histidine-alanine-valine (HAV) sequence to form a functionalized hydrogel; chemically attaching a multivalent PEG succinimidyl glutarate to the functionalized hydrogel; and subjecting the resulting hydrogel to a crosslinking reaction, thereby causing the hydrogel to crosslink.

In another aspect, the present disclosure provides a biomaterial produced by the preparation method disclosed herein. The produced biomaterial may be isolated or purified using known techniques before use.

E. Physical Properties

The biomaterial may have a stiffness of about 500 Pa to about 10 kPa. The stiffness may be at least 600 Pa, at least 800 Pa, at least 2 kPa, at least 4 kPa, at least 6 kPa, or at least 8 kPa. The stiffness may be less than 9 kPa, less than 7 kPa, less than 5 kPa, less than 3 kPa, or less than 1 kPa. In some embodiments, the biomaterial has a stiffness of about 800 Pa to about 5 kPa, such as about 1 kPa, about 2 kPa, about 3 kPa, or about 4 kPa. A desired stiffness may be achieved, for example, by changing the crosslinker (such as HPA) concentration. The crosslinker concentration may be varied by adjusting (1) the starting concentration of the crosslinker when conjugating to the hydrogel (such as gelatin), and/or (2) the time allowed for conjugating to the hydrogel.

The biomaterial may include a plurality of pores. The pores may be interconnected to form a porous network throughout the hydrogel and biomaterial thereof. Each individual pore may have a diameter of about 10 μm to about 200 μm. Each individual pore may be at least 20 μm, at least 40 μm, at least 60 μm, at least 80 μm, at least 100 μm, at least 120 μm, at least 140 μm, at least 160 μm, or at least 180 μm. Each individual pore may be less than 190 μm, less than 170 μm, less than 150 μm, less than 130 μm, less than 110 μm, less than 90 μm, less than 70 μm, less than 50 μm, or less than 30 μm. In some embodiments, the biomaterial includes a plurality of pores, each individual pore having a diameter of about 20 μm to about 80 μm, such as about 30 μm, about 50 μm, or about 70 μm.

### 3. METHODS

The present disclosure also provides methods of promoting large blood vessel growth, or arteriogenesis, for treating a disorder in a subject. The method may include administering to an area of the subject in need thereof a therapeutically effective amount of the biomaterial. The description of the biomaterials, hydrogels, peptides, crosslinking, and preparation can also be applied to the methods disclosed herein.

As mentioned above, the method promotes arteriogenesis. Arteriogenesis refers to the growth or development of collateral arteries or arterioles. Thus, the biomaterial and method thereof can provide artery growth, arteriole growth, or a combination thereof in the area of administration. Artery growth, arteriole growth, or a combination thereof refers to sprouting of new arteries or arterioles from pre-existing arteries or arterioles, as well as the transformation of smaller, pre-existing vessels into arteries or arterioles. The growth can also occur and/or extend beyond the area of administration. The artery growth, arteriole growth, or a combination thereof can occur within about 1 day to about 14 days. Growth can also continue past this timeframe.

The artery growth, arteriole growth, or a combination thereof can include growth of a single vessel or a plurality of vessels in the area of administration. The growth of artery(s), arteriole(s), or both can correspond to providing a vessel having a diameter of about 0.05 mm to about 1 mm, such as about 0.1 mm to about 1 mm, about 0.2 mm to about 0.9 mm, about 0.25 mm to about 0.85 mm, about 0.3 mm to about 0.8 mm, about 0.4 mm to about 0.95 mm, or about 0.5 mm to about 1 mm.

With the artery growth, arteriole growth, or both, an associated increase in blood flow can occur. For example, the biomaterial and method thereof can increase blood flow in the area of administration compared to that same area prior to administration. The increase in blood flow can be measured by perfusion index. Perfusion index refers to the rate of blood flow in a compromised tissue relative to a control tissue. Perfusion index can be measured by techniques such as laser doppler imaging or pulse oximetry. In some embodiments, the increase in blood flow in the area of administration can be 1.1× greater, 1.2× greater, 1.3× greater, 1.4× greater, 1.5× greater, 1.6× greater, 1.7× greater, 1.8× greater, 1.9× greater, 2× greater, 2.5× greater, or 3× greater compared to that same area prior to administration. The increase in blood flow can be measured at 1 day, 2 days, 3 days, 4 days, 5 days, 6 days, 7 days, 8 days, 9 days, 10 days, 11 days, 12 days, 13 days, or 14 days post-administration.

The biomaterial and method thereof can also repair damage caused by ischemia in the subject. The repair of damage can be measured by Tarlov score, ischemia score, modified ischemia score, or a combination thereof. These scores have been described herein in the examples and the figures. In some embodiments, the repair in damage caused by ischemia is measured by modified ischemia score. In some embodiments, the biomaterial and method thereof repairs damage caused by ischemia in the subject as measured by a modified ischemia score of above 5, above 6, or 7. In some embodiments, the biomaterial and method thereof repairs damage caused by ischemia in the subject as measured by a modified ischemia score of 7.

The biomaterials may be administered in a variety of different anatomical locations or areas of the subject depending on, e.g., the disorder being treated. The area of administration may be any area that is in need of increased blood flow. For example, the area of administration may be an area having impaired blood flow (e.g., suffering from ischemia). In addition, the area of administration may be an area adjacent to an area of the subject suffering from ischemia. In some embodiments, the biomaterial is implanted in an extremity of a subject suffering from ischemia in that extremity, such as found in peripheral artery disease. In some embodiments, the biomaterial is used to contact or coat existing arteries in the subject in order to induce collateral vessel growth and blood flow. In some embodiments, the biomaterial is implanted in an area that has no existing artery(s) or arteriole(s) present. Accordingly, the disclosed biomaterials may induce arteriogenesis from preexisting vasculature, induce arteriogenesis de novo, or both.

The method may be used for treating a number of different disorders that can benefit from increased blood flow. Examples include, but are not limited to, ischemic heart disease, coronary artery disease, ischemic heart failure, peripheral artery disease, critical limb ischemia, diabetic foot ulcer, organ transplant, wounds, and bone fracture. In some embodiments, the disorder is selected from the group consisting of ischemic heart disease, coronary artery disease, ischemic heart failure, peripheral artery disease, critical limb ischemia, and diabetic foot ulcer. In some embodiments, the disorder is selected from the group consisting of peripheral artery disease, critical limb ischemia, and diabetic foot ulcer. In some embodiments, the disorder is peripheral artery disease.

The subject that can benefit from the disclosed methods can be any type of mammal. As used herein, the term subject, includes humans and mammals (e.g., mice, rats, pigs, cats, dogs, and horses). Typical subjects of the present disclosure may include mammals, particularly primates, and especially humans. For veterinary applications, suitable subjects may include, for example, livestock such as cattle, sheep, goats, cows, swine, and the like; poultry such as chickens, ducks, geese, turkeys, and the like, as well as domesticated animals particularly pets such as dogs and cats. For diagnostic or research applications, suitable subjects may include mammals, such as rodents (e.g., mice, rats, hamsters), rabbits, primates, and swine such as inbred pigs and the like. In some embodiments, the subject is human.

As mentioned above, the biomaterial may be administered with cells, molecules, or both that aid in promoting arteriogenesis. For example, the biomaterial may further include, but is not limited to, a plurality of cells and proteins. In some embodiments, the biomaterial is administered without any cells, proteins, or without both.

In another embodiment, tissue may be embedded in the biomaterial as described herein. The tissue may be mammalian tissue, fish tissue, reptilian tissue, bird tissue, amphibian tissue, or arthropod tissue. In another embodiment, the tissue may be human tissue or mouse tissue. In a further embodiment, the tissue may be brain tissue, lung tissue, stomach tissue, bladder tissue, liver tissue, kidney tissue, skin tissue, or any mammalian organ tissue known in the art.

A suitable density of the plurality of cells as described herein to be provided to the biomaterial may be at least about 0.1×10⁵ cells/cm², at least about 0.2×10⁵ cells/cm², at least about 0.3×10⁵ cells/cm², at least about 0.4×10⁵ cells/cm², at least about 0.5×10⁵ cells/cm², at least about 0.6×10⁵ cells/cm², at least about 0.7×10⁵ cells/cm², at least about 0.8×10⁵ cells/cm², at least about 0.9×10⁵ cells/cm², at least about 1×10⁵ cells/cm², at least about 1.1×10⁵ cells/cm², at least about 1.2×10⁵ cells/cm², at least about 1.3×10⁵ cells/cm², at least about 1.4×10⁵ cells/cm², at least about 1.5×10⁵ cells/cm², at least about 1.6×10⁵ cells/cm², at least about 1.7×10⁵ cells/cm², at least about 1.8×10⁵ cells/cm², at least about 1.9×10⁵ cells/cm², or at least about 2.0×10⁵ cells/cm².

A. Administration

The disclosed biomaterials may be incorporated into pharmaceutical compositions suitable for administration to a subject well known to those skilled in the pharmaceutical art. The pharmaceutical composition may be prepared for administration to a subject. Such pharmaceutical compositions can be administered in dosages and by techniques well known to those skilled in the medical arts taking into consideration such factors as the age, sex, weight, and condition of the particular subject, and the route of administration.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate: powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols, such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator. The route by which the composition is administered and the form of the composition will dictate the type of carrier to be used.

The composition can be administered prophylactically or therapeutically. The compositions can be administered by methods well known in the art as described in Donnelly et al. (Ann. Rev. Immunol. 1997, 15, 617-648); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), which are all incorporated by reference herein in their entirety. One skilled in the art would know that the choice of a pharmaceutically acceptable carrier, including a physiologically acceptable compound, depends, for example, on the route of administration. The compositions can be delivered via a variety of routes. A typical delivery route includes parenteral administration, such as intradermal, intramuscular, intraarterial or subcutaneous delivery.

### 4. EXAMPLES

The hydrogels disclosed in the Examples may be referred to as GelMA-Cad, Cad, or GelCad depending on the type of crosslinker/crosslinking. For example, GelMA-Cad refers to methacrylated gelatin including peptide, while Cad or GelCad typically refer to gelatin including peptide but without methacrylate groups (e.g., crosslinked through a different mechanism). Negative control hydrogels, which contain a scrambled peptide in place of the N-cadherin peptide, may be referred to as GelMA-Scram, Scram, or GelScram depending on the type of crosslinker/crosslinking.

### Example 1. Synthesis and Characterization of GelMA Functionalized with N-Cadherin Peptide

### Materials & Methods

Ge/MA synthesis and characterization. Methacrylated gelatin (GelMA) was synthesized as described previously (Loessner et al., Nat. Protoc. 2016, 11, 727). Type A porcine skin gelatin (Sigma) was mixed at 10% (w/v) into DI water (sourced from an in-house Continental Modulab ModuPure reagent grade water system) at 60° C. and stirred until fully dissolved. Methacrylic acid (MA) (Sigma) was slowly added to the gelatin solution and stirred at 50° C. for 3 hours. The solution was then centrifuged at 3,500×g for 3 minutes and the supernatant was collected. Following a 5× dilution with additional warm (40° C.) UltraPure water (Thermo Fisher) to stop the reaction, the mixture was dialyzed against DI water for 1 week at 37° C. using 12-14 kDa cutoff dialysis tubing (Fisher) to remove salts and MA. The pH of the solution was then adjusted to 7.35-7.45 by adding HCl or NaOH as measured with a Thermo Fisher Scientific Orion Star pH meter. The resulting GelMA solution was lyophilized for 3 days using a Labconco lyophilizer and stored at −20° C.

Peptide conjugation and characterization. Peptides were conjugated to GelMA as previously reported (Bian et al., Proc. Natl. Acad. Sci. 2013, 110, 10117) with slight modifications. Briefly, GelMA was reconstituted in triethanolamine (TEOA) buffer (Sigma) to create a 10% w/v solution and stirred at 37° C. for 2 hours until fully dissolved. The pH of the solution was then adjusted to 8.0-8.5 using HCl or NaOH. Scrambled (Ac-AGVGDHIGC, to make GelMA-Scram) or N-Cadherin mimic (Ac-HAVDTGGGC, to make GelMA-Cad) peptides (GenScript) were added to the GelMA/TEOA buffer to form a 1% w/v solution. The cysteine residue at the C-terminal end of the peptides permitted a Michael-type addition reaction with GelMA. The solution was stirred at 37° C. for 24 hours and then dialyzed against DI water using 6-8 kDa cutoff dialysis tubing (Spectrum) for 1 week at 37° C. The pH of the solution was then adjusted to 7.35-7.45 using HCl or NaOH, and the solution was lyophilized and stored at −20° C. Conjugation was verified through 1H-NMR using a Bruker 500 Hz NMR spectrometer set to 37° C. for the presence of the amino acid valine.

An alternative process for conjugating a peptide to the gelatin backbone of GelMA may be used as follows: GelMA is reconstituted in triethanolamine buffer to create a 10% solution, and stirred at 37° C. for 2 hours until fully dissolved. The pH is adjusted between 8-8.5. The peptide is then added to the biomaterial (between 0.1%-5% weight/volume), and the mixture is stirred at 37° C. for 24 hours. The solution is then filtered and dialyzed using a tangential flow filtration system (2 kDa pore size).

Fourier-transform infrared spectroscopy. 198 mg of potassium bromide (Sigma) was added to 2 mg of lyophilized gelatin, GelMA, GelMACad, or GelMA-Scram and crushed using a mortar and pestle. The crushed samples were transferred to a 13 mm Specac evacuable pellet press die and compressed into a thin disc using a Specac manual hydraulic press. An additional disc was made using only potassium bromide for calibration. Samples were stored in a dry container overnight and analyzed the following day using a Bruker Tensor 27.

Atomic force microscopy. GelMA, GelMA-Scram, and GelMA-Cad were reconstituted and polymerized into hydrogel discs as described in the cell seeding section herein. A Bruker Dimension Icon Atomic Force Microscope was used to measure hydrogel stiffness. 0.01 N/m Novascan probes containing a 4.5 μm polystyrene bead (PT.PS.SN.4.5.CAL) were used to measure three distinct 5×5 μm areas of each hydrogel. Three hydrogel disc replicates of each sample were included for a total of 576 stiffness measurements per sample. For each individual force curve, a first order baseline correction was performed, and the Hertzian model was used to calculate Young's modulus. For tool calibration, polyacrylamide hydrogels were prepared as previously reported (Stroka, et al., Blood 2011, 118, 1632) and measured prior to GelMA and its derivatives.

Scanning electron microscopy. Lyophilized GelMA, GelMA-Cad, and GelMA-Scram were reconstituted in PBS to form 10% (w/v) solutions with 0.05% LAP initiator (Sigma). 30 μL of each hydrogel solution was added to a Ted Pella pin mount and crosslinked by an 8 second exposure to a 25 mW/cm² UV light using a ThorLabs UV Curing LED System. These pin mounts were stored in a Ted Pella mount storage tube and then placed in a −80° C. freezer overnight. The following day, the samples were transferred to a Labconco lyophilizer for an additional 2 days and then stored at room temperature until used. To characterize the internal microstructures of GelMA, GelMA-Cad and GelMA-Scram, the dried samples were observed using a scanning electron microscope (Zeiss Merlin) at an accelerating voltage of 2 kV. ImageJ software was used to quantify pore sizes, where the mean diameter of each pore was considered the average pore size.

### Results & Discussion

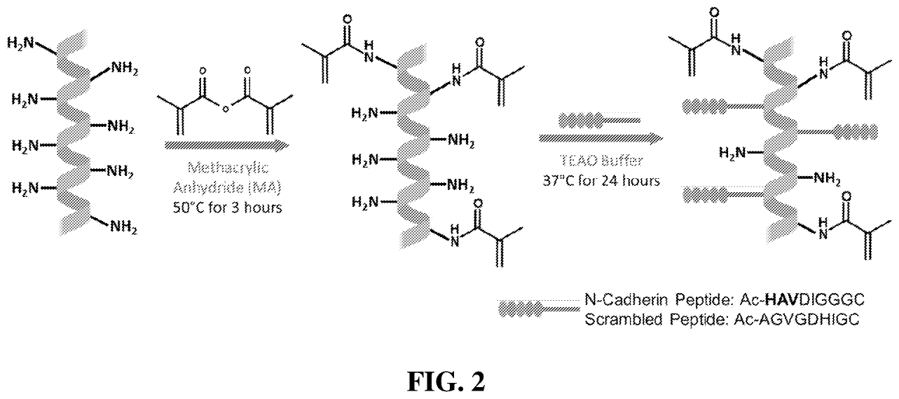

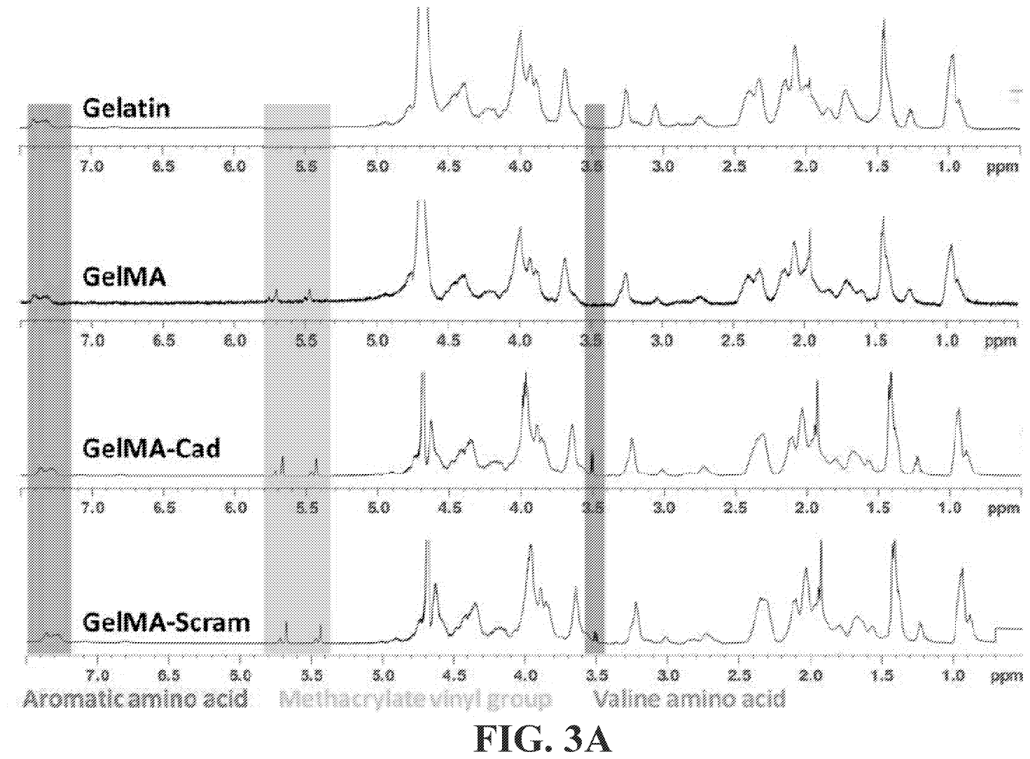

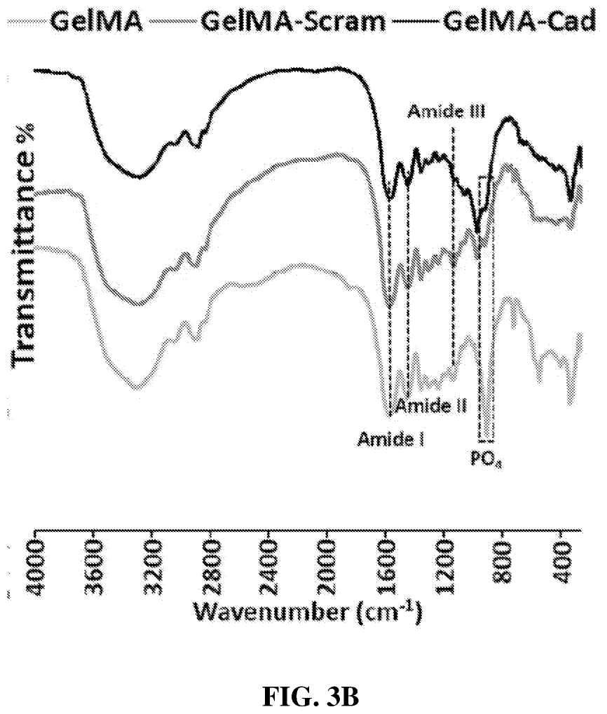

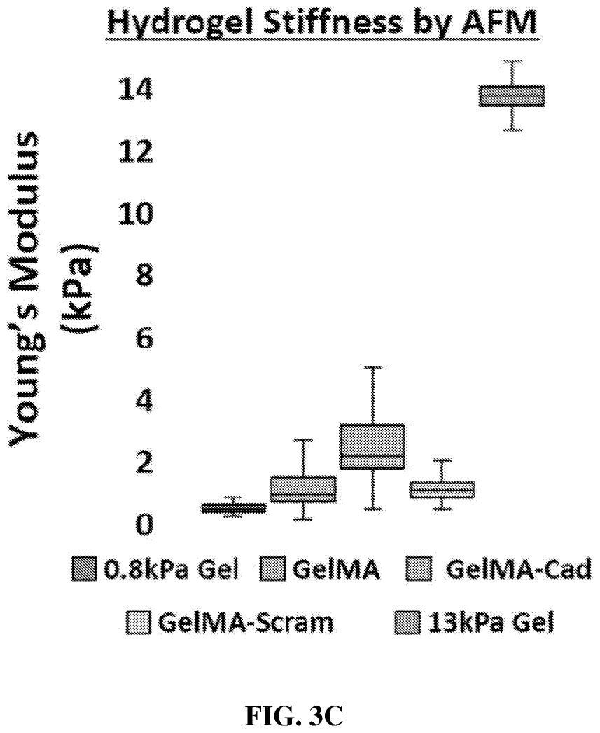

GelMA was chosen as a base material due to its ease of handling and robust mechanical properties (after crosslinking) compared to ECMs such as Matrigel and HA. To generate the GelMA-Cad scaffold, porcine gelatin was first functionalized with methacrylic anhydride in order to create the GelMA backbone that could be crosslinked when exposed to the photoinitiator LAP and UV light (FIG. 2). This modification was confirmed through the presence of methacrylic side chain protons (˜5.45 and 5.7 ppm) using ¹H-NMR (FIG. 3A). GelMA was then functionalized with the extracellular epitope of N-cadherin (HAVDIGGGC) to prepare GelMA-Cad, or with an N-cadherin-scrambled peptide (AGVGDHIGC) to prepare GelMA-Scram. The conjugation of these peptides to the scaffold was also confirmed via ¹H NMR through the presence of valine protons (˜3.5 ppm), which are not present in the gelatin or GelMA spectra (FIG. 3A). Additionally, Fourier-transform infrared spectroscopy (FTIR) was employed to further validate successful functionalization. The FTIR transmittance spectra showed a noticeable decrease in PO₄ peaks (1000 cm-1) and amide peaks I, II, III (1640, 1540, and 1250 cm⁻¹, respectively) in GelMA-Cad and GelMA-Scram samples compared to GelMA (FIG. 3B), likely due to peptide conjugation. Collectively, these data suggest GelMA was properly synthesized and functionalized.

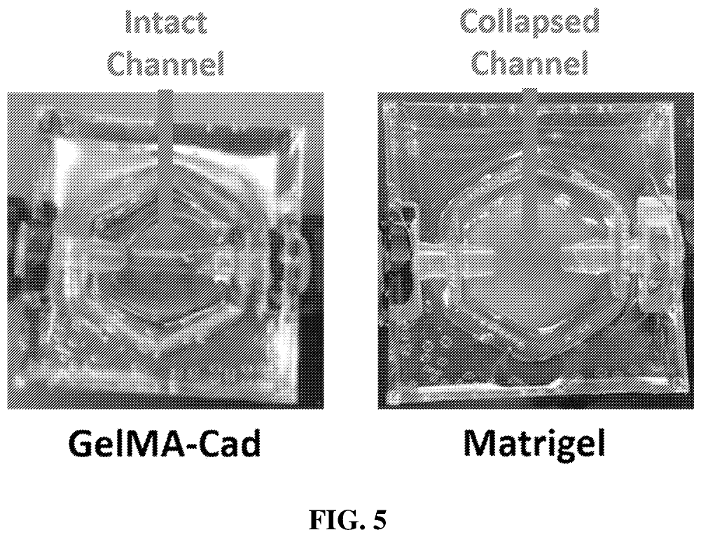

The mechanical and physical properties of the crosslinked hydrogels were studied. In order to determine the stiffness of GelMA, GelMA-Cad, and GelMA-Scram, atomic force microscopy (AFM) was performed. 0.8 kPa and 13 kPa polyacrylamide hydrogels were produced and measured by AFM to validate that the tool was properly calibrated. After crosslinking with LAP and UV light. GelMA, GelMA-Cad, and GelMA-Scram exhibited stiffness values of approximately 1-5 kPa (FIG. 3C). Despite its relatively low elastic modulus, GelMA-Cad is stiff enough to maintain patterned architectures: when it was crosslinked around silicone tubing, followed by manual extraction of the tubing, a straight, a perfusable channel remained in the GelMA-Cad (FIG. 5), whereas Matrigel collapses and the perfusion channel does not remain patent (FIG. 5). Thus, similar to GelMA, GelMA-Cad can be patterned into more complex structures.

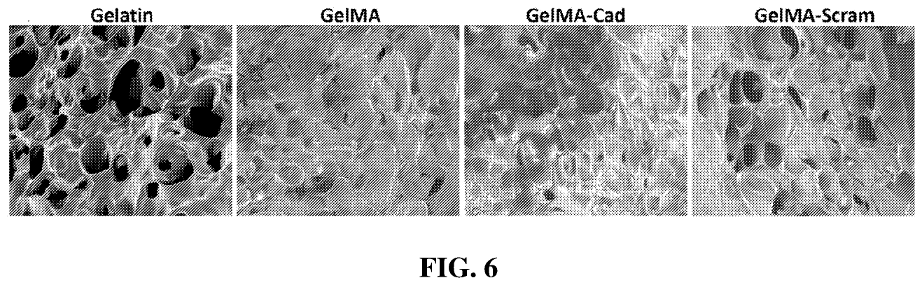

The microstructure of the hydrogels was characterized by scanning electron microscopy (SEM). Porous network structures are commonly observed in hydrogels and are important for nutrient diffusion, cell integration and removal of waste products, and the degree of chemical substitution has an inverse relation to pore size upon crosslinking. The average pore size diameter of GelMA, GelMA-Cad, and GelMA-Scram were measured at 42.8±0.2, 43.1±0.2, and 42.4±0.2 μm, respectively (FIG. 6). These measurements confirm that the hydrogels all have similar physical and mechanical properties.

### Example 2. Arteriogenesis Experiment—1

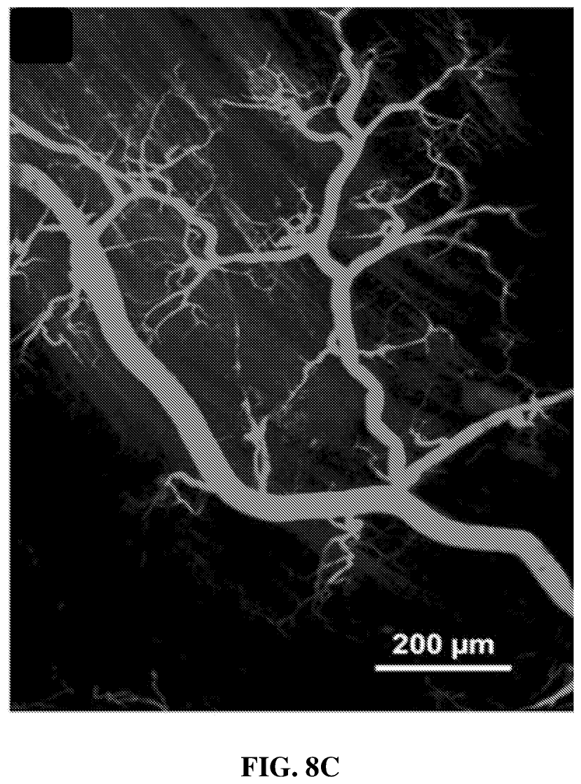

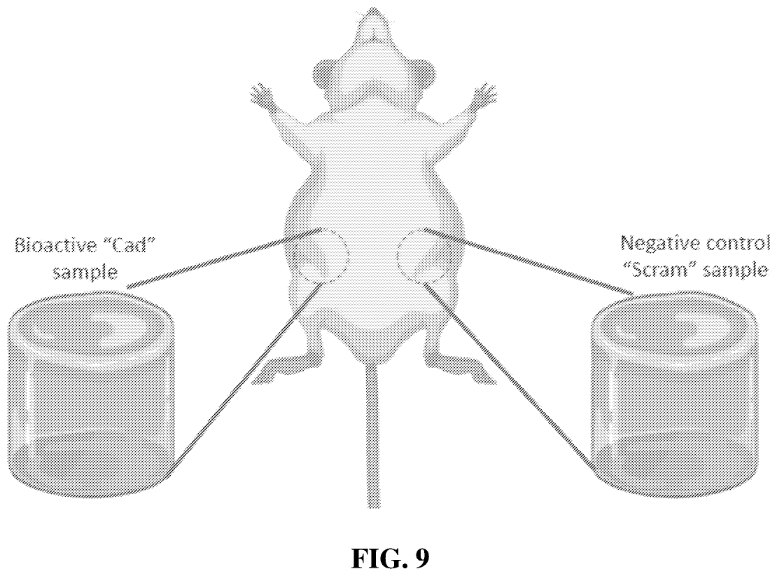

The hydrogel was prepared as described in Example 1. As shown in FIG. 9, hydrogels were cast in 5 mm×5 mm cylinders and implanted into the fat pad of mice. After 17 days, mice received a tail vein injection of DiI, a red lipophilic dye that inserts into cell membranes. Mice were then euthanized by perfusion with paraformaldehyde, and the hydrogel plugs were removed, subjected to a tissue clearing protocol, labeled with other antibodies or dyes, and imaged. Thus, if newly formed blood vessels are connected to the host circulatory system, they will turn red.

Negative control Scram sample: Fat pad and gel are demarcated based on DAPI stain, which labels cell nuclei—there are clearly minimal cells in the hydrogel plug and many cells in the fat pad. The green label is for ZO-1, a tight junction protein expressed in many different cell types, including endothelial cells. There are no obvious red cells. It appears that there is one small vessel growing into the hydrogel that may express ZO-1 (green signal). However, there is no obvious red signal except near the top of the fat pad.

Bioactive Cad sample: On the fat pad side, we were able to find a perfused arteriole (red signal; green is ZO-1 and blue is DAPI). According to the literature, the fat pad has low arteriole density, where any arterioles present are very small (typically 40 μm or smaller) and most vessels are capillaries. These DiI images are taken ˜3 millimeters deep into the plug. The vessels clearly have arteriole size and architecture.

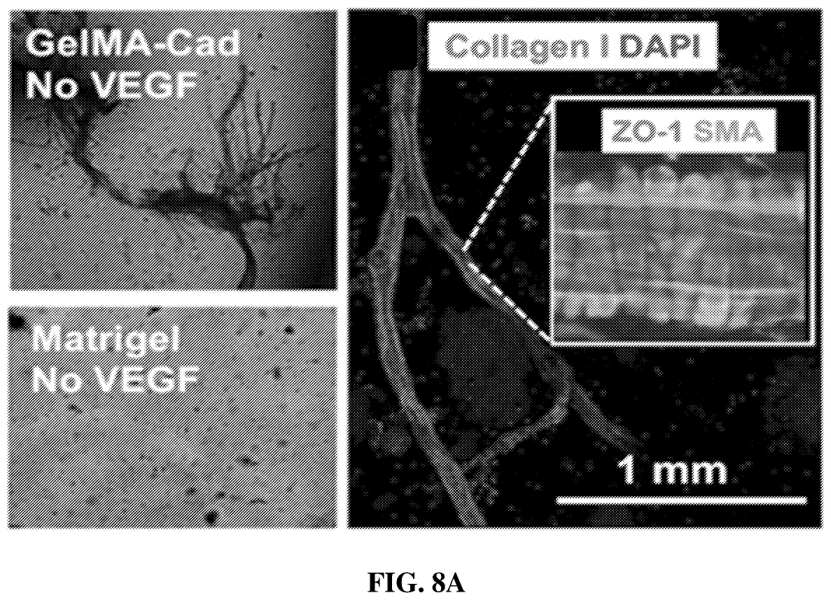

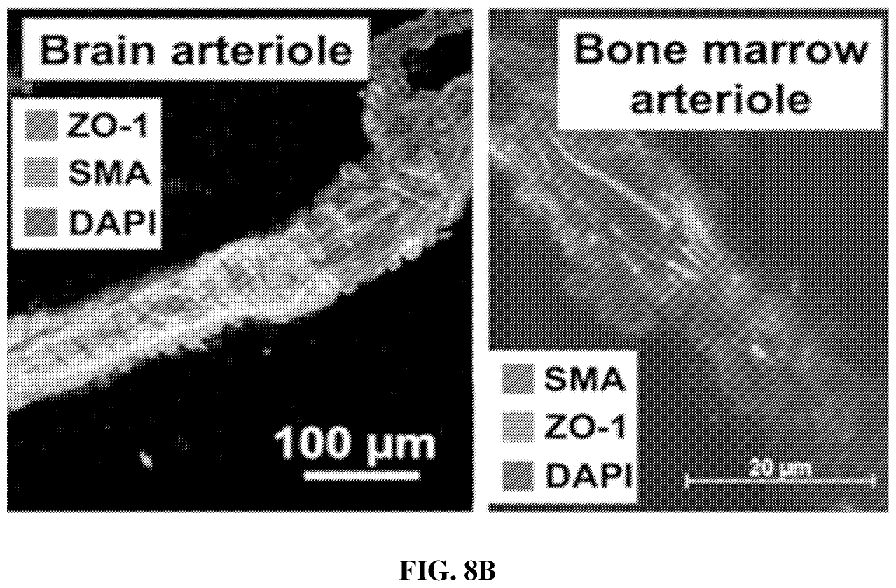

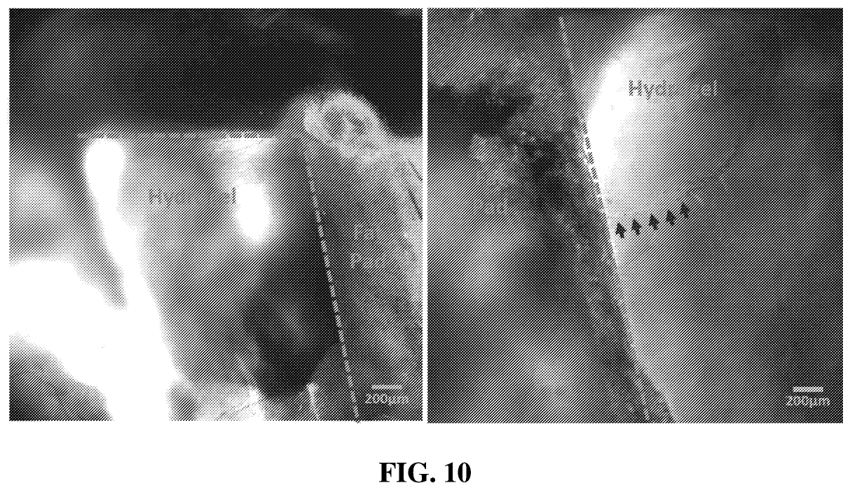

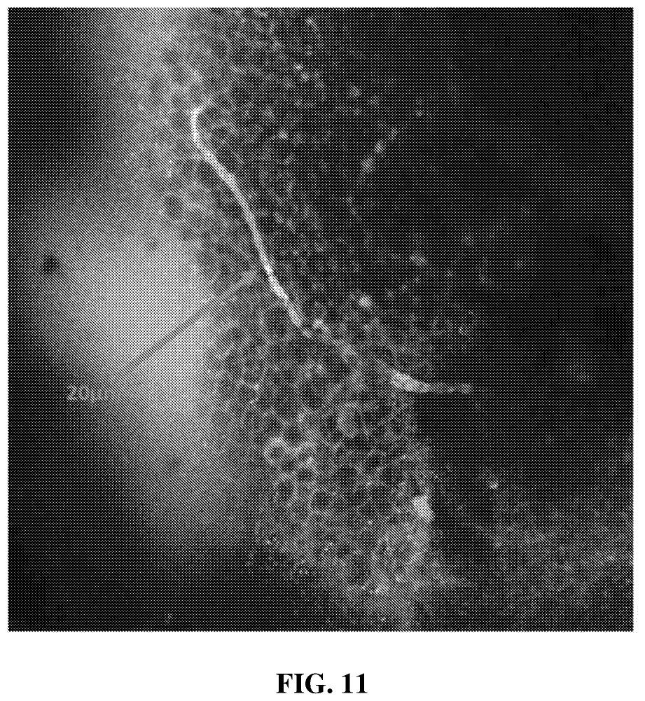

Images of the controls show negligible large blood vessel growth (FIG. 10). In contrast, images of bioactive Cad showed vessels having arteriole size and architecture (FIG. 8C, FIG. 11, and FIG. 12).

### Example 3. Arteriogenesis Experiment—2

Materials & Methods. Gelatin was functionalized with peptides using EDC/NHS chemistry and crosslinked with transglutaminase. We utilized a peptide mimicking the first extracellular epitope of type 1 cadherins or a scrambled control, noted as “GelCad” or “GelScram.” To assess arteriogenesis in an ex vivo system, mouse or human cortical brain tissue was manually dissociated and embedded in hydrogels comprised of GelCad or Matrigel. After 4 days, brightfield and fluorescence microscopy was used to assess vascular outgrowth. For in vivo studies, GelCad and GelScram hydrogels were embedded subcutaneously in C57BL/6 mice. At various intervals, hydrogels were excised, and arteriole infiltration was analyzed by brightfield and fluorescence microscopy. In some cases, mice received a tail vein injection of DiI 30 minutes prior to euthanasia to assess perfusion of the hydrogel by host circulation. All animal protocols were approved by the Vanderbilt University IACUC.

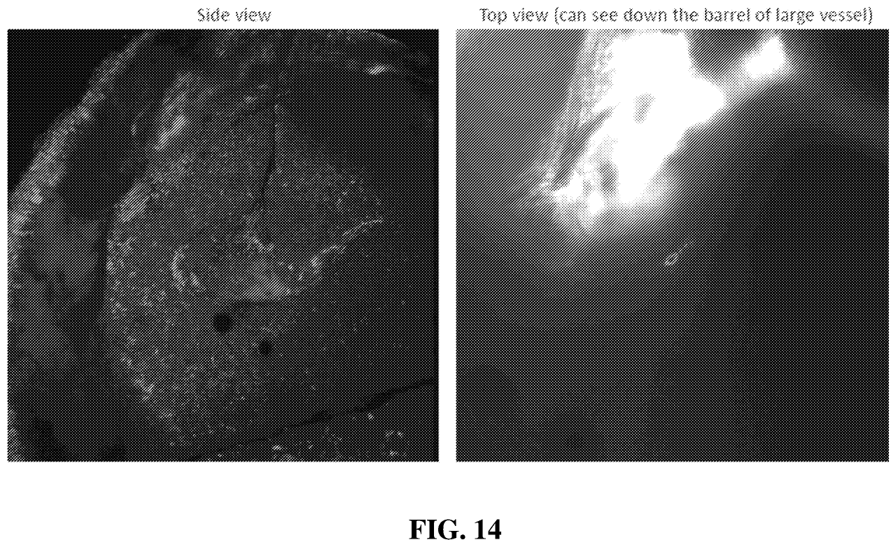

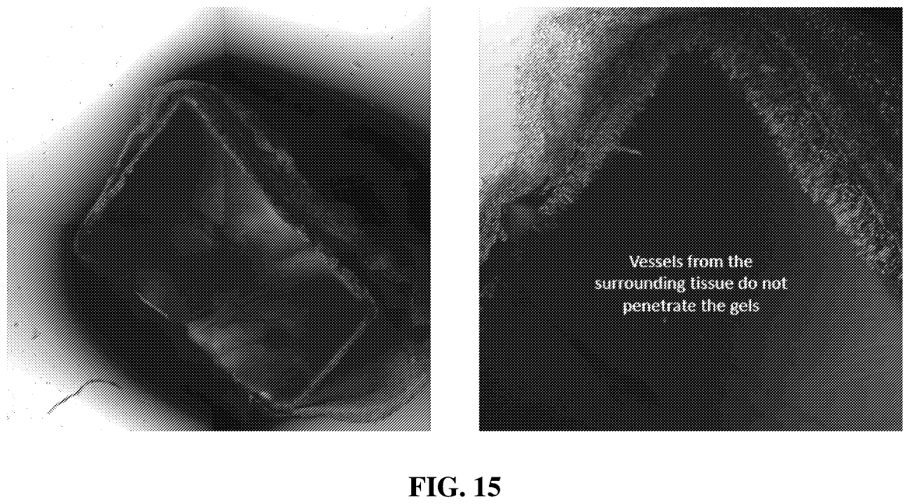

Results & Discussion. We embedded primary mouse brain tissue into GelCad hydrogels. The tissue grew tubular structures resembling blood vessels. Fluorescence microscopy identified these tubular structures as arterioles with biomimetic architecture including concentric rings of a-smooth muscle actin positive (a-SMA+) smooth muscle and a basement membrane of collagen I surrounding an inner layer of VE-cadherin+ endothelial cells. This arteriole growth was only observed in GelCad but not Matrigel hydrogels. Next, we embedded GelCad and GelScram hydrogels as pre-cast hydrogels in mouse fat pads. GelScram hydrogels showed no vascular infiltration after 2 weeks, whereas GelCad hydrogels have extensive arteriole networks throughout the entire ˜100 mm³ plug. After DiI injection, there was little evidence of dye penetration for GelScram, whereas GelCad hydrogels have extensive dye labeling in the vessel structures indicating anastomosis to host vasculature. Representative images can be seen at FIG. 8A, FIG. 8B, FIG. 12, FIG. 14, and FIG. 15.

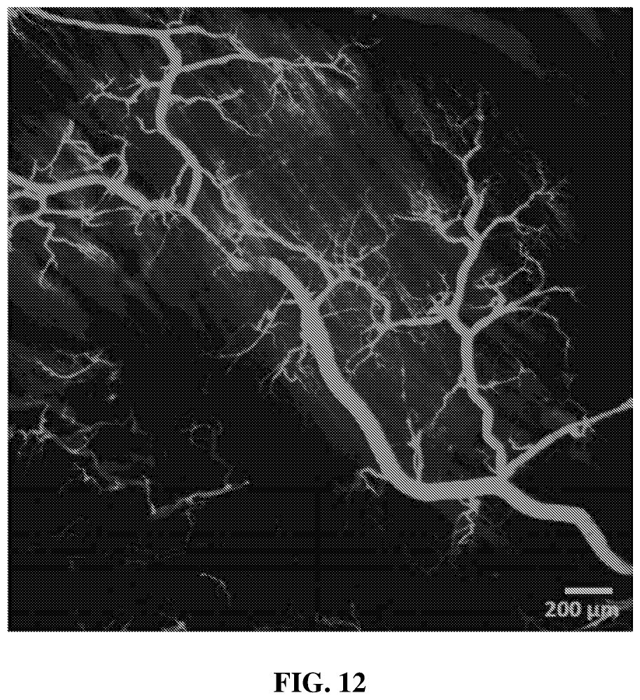

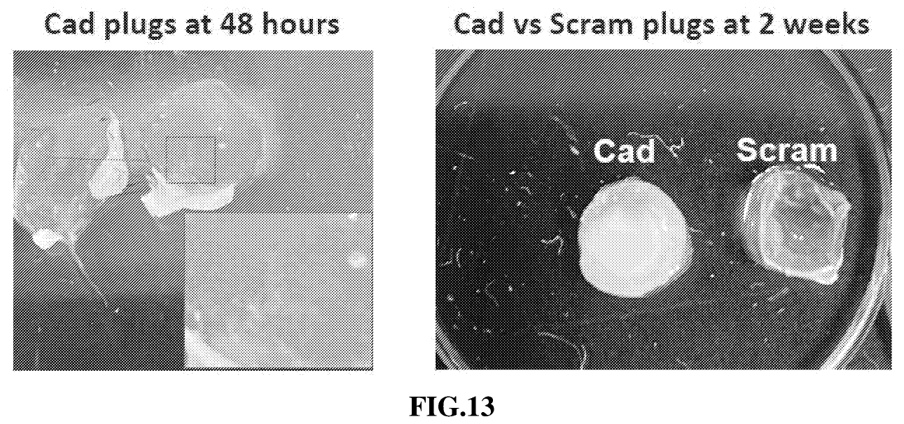

In addition, GelCad plugs were excised and imaged at 48 hours and 2 weeks post implantation. GelCad hydrogels turn white whereas Scram hydrogels remain clear. This is hypothesized to be due to cellular infiltration and remodeling of the hydrogels, further bolstered by identification of large, branching architectures at 48 hours. This can be seen at FIG. 13.

### Example 4. Arteriogenesis Experiment—3

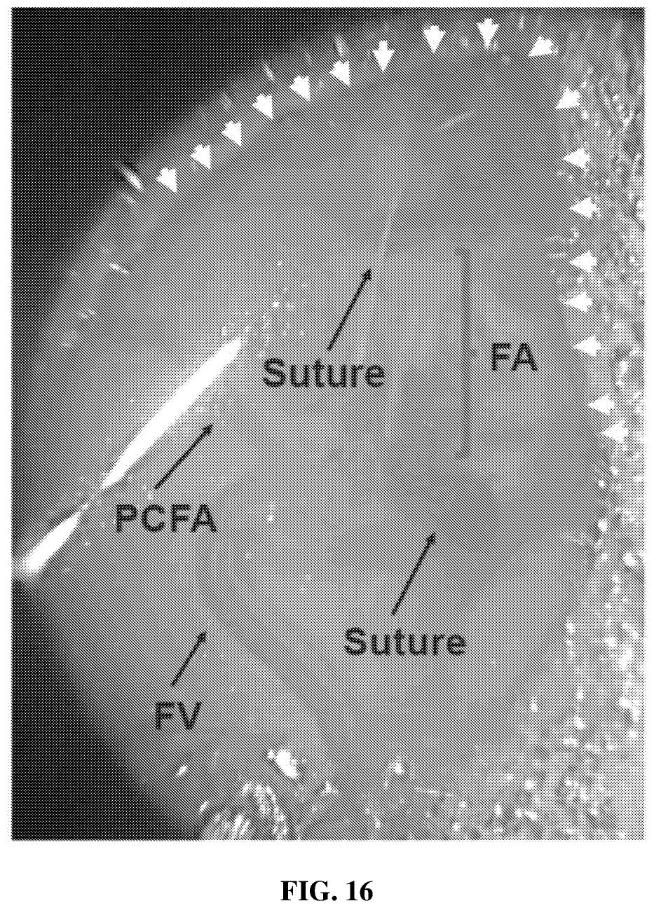

Materials & Methods. Gelatin was functionalized with peptides using EDC/NHS chemistry. We utilized a peptide mimicking the first extracellular epitope of type 1 cadherins or a scrambled control, noted as “GelCad” or “GelScram.” Hydrogels were formed by crosslinking with microbial transglutaminase (mTG) or 4-arm polyethylene glycol succinimidyl glutarate (PEG-SG) having a number average molecular weight of 20 kDa. Gelation time and stiffness of PEG-SG-crosslinked hydrogels was measured using inversion tests and rheometry. For in vivo studies on vascular growth, GelCad and GelScram hydrogels were embedded subcutaneously in C57BL/J6 or BALB/c mice. At various intervals, hydrogels were excised, and arteriole infiltration was analyzed by brightfield and fluorescence microscopy. In some cases, mice received a retro-orbital injection of microbubbles followed by ultrasound imaging. For in vivo studies on the therapeutic potential of the hydrogels, BALB/c mice were subjected to femoral artery ligation (FIG. 16), followed by deposition of GelCad or GelScram hydrogels in the subcutaneous space before closure of the incision. Gross appearance of the surgical limb, as well as ambulation, was quantified over time using Tarlov and Ischemia Scores. Laser doppler imaging was used to measure blood flow over time. After 14 days, animals were euthanized and hindlimbs were sectioned and analyzed by an expert histopathologist who was blinded to the identity of the hydrogels. All animal protocols were approved by the Vanderbilt University IACUC.

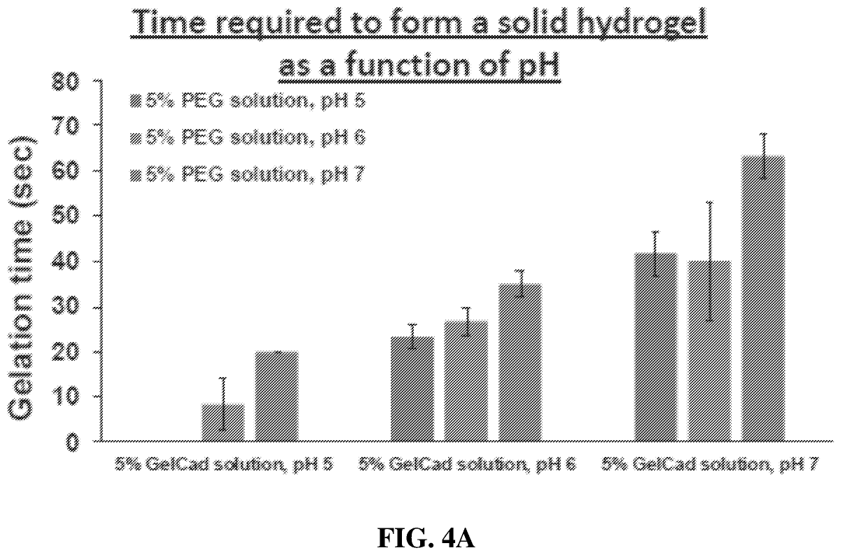

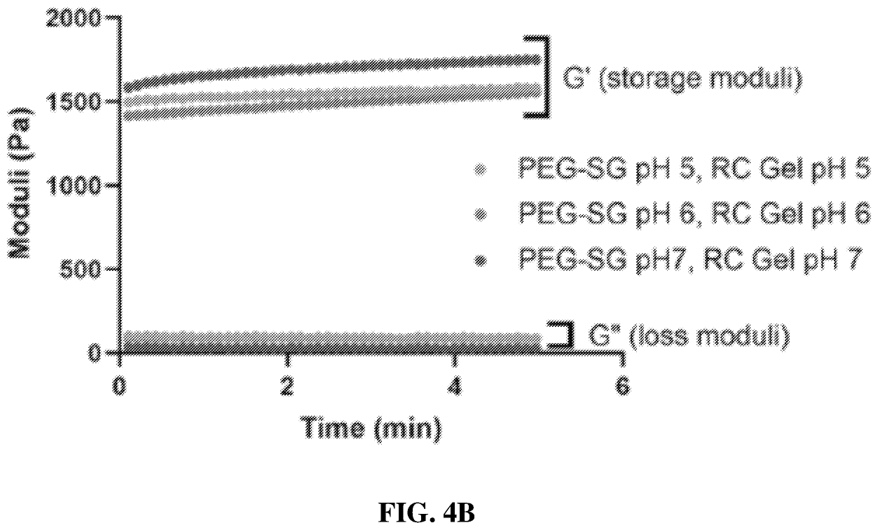

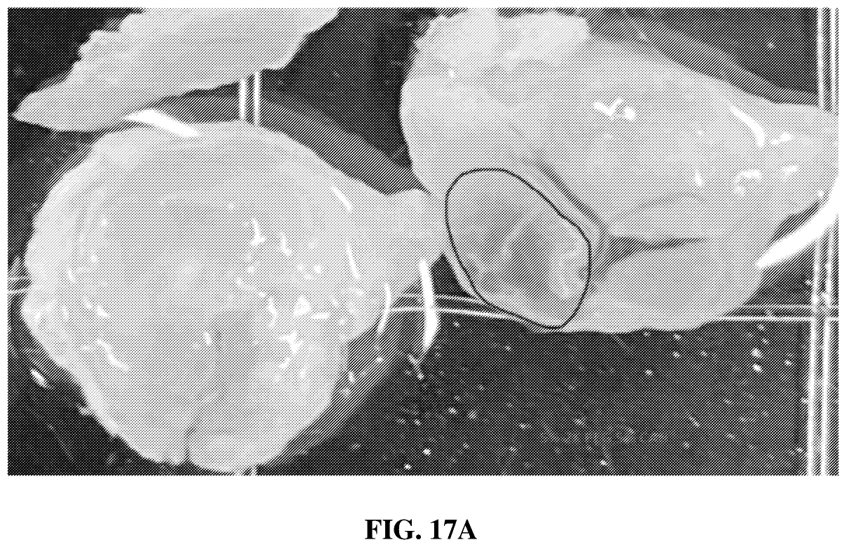

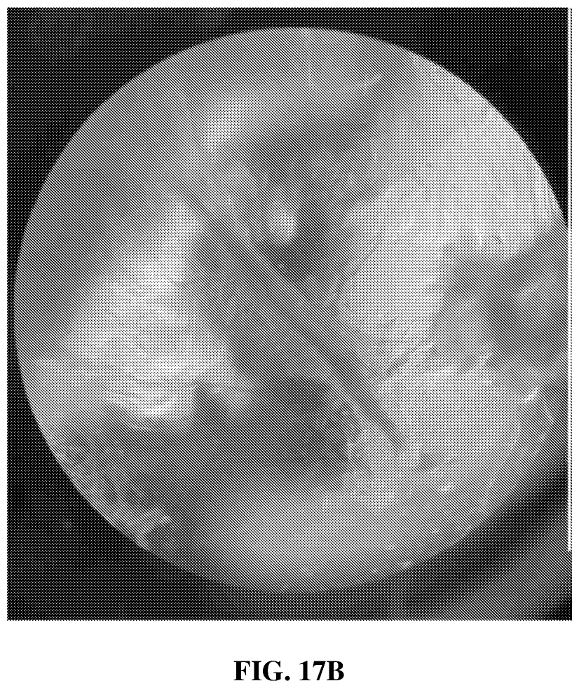

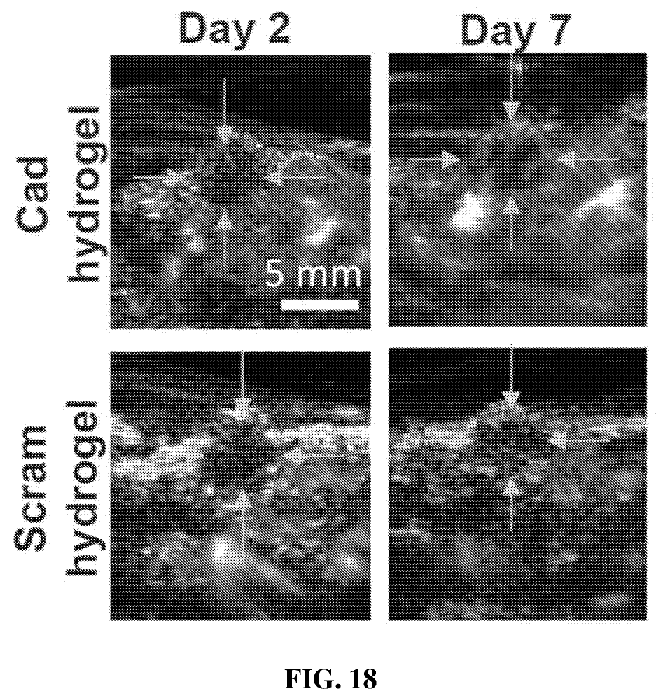

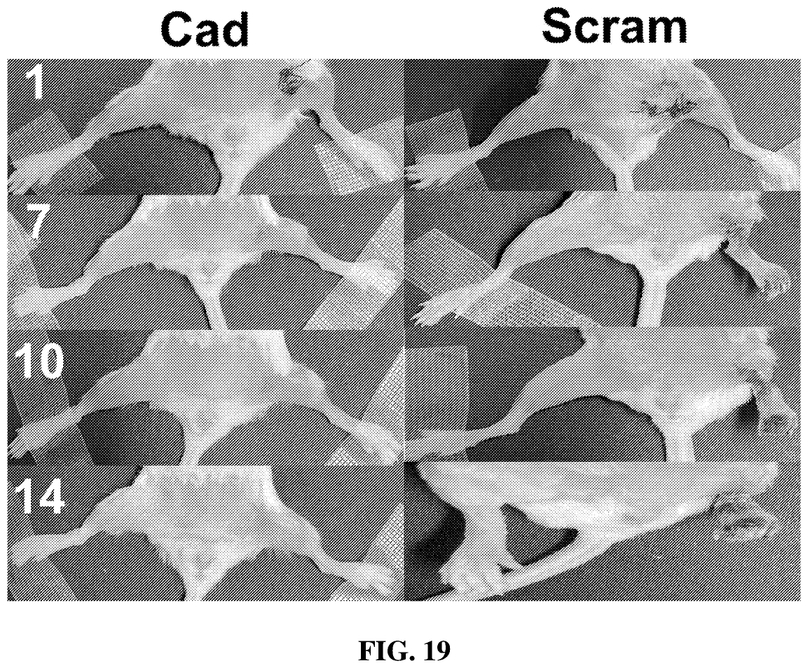

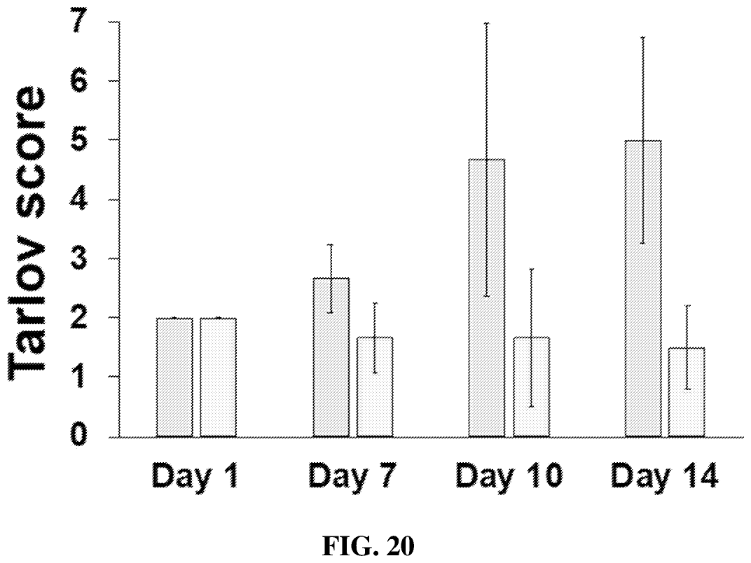

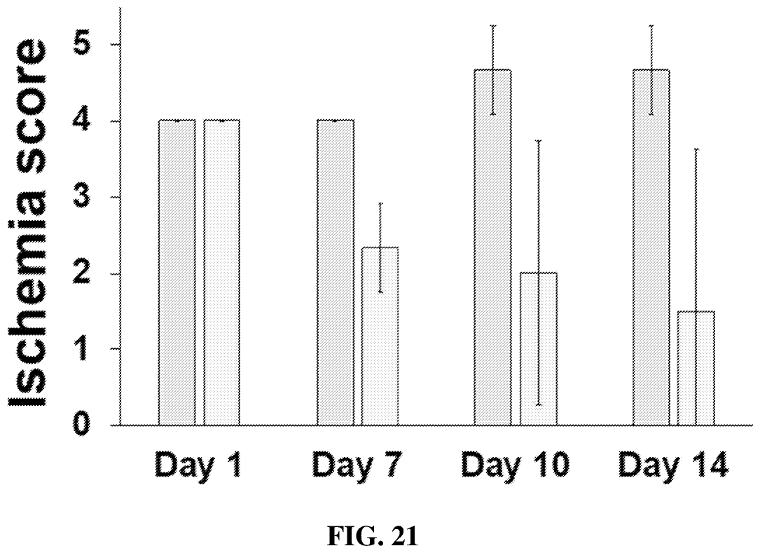

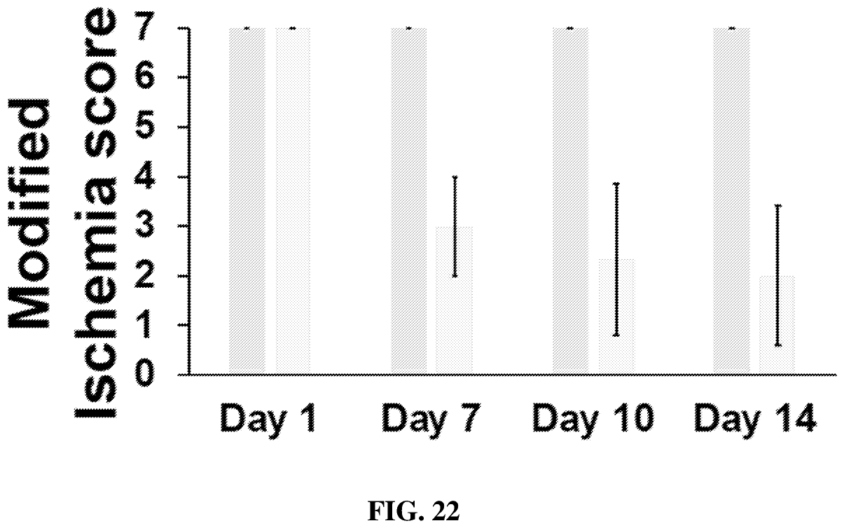

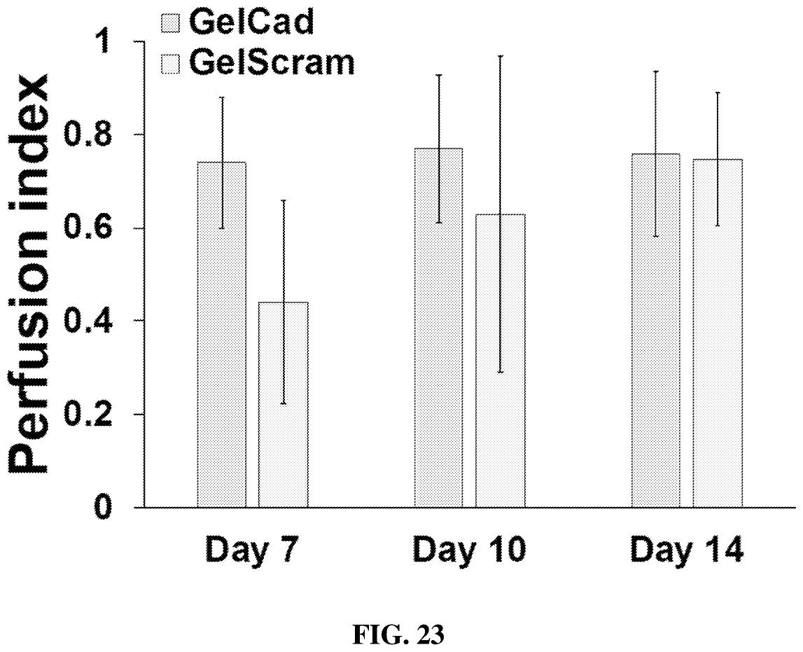

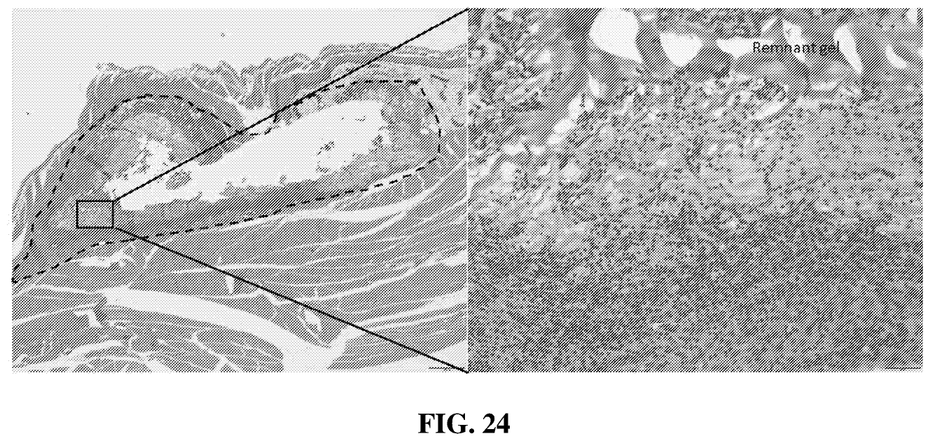

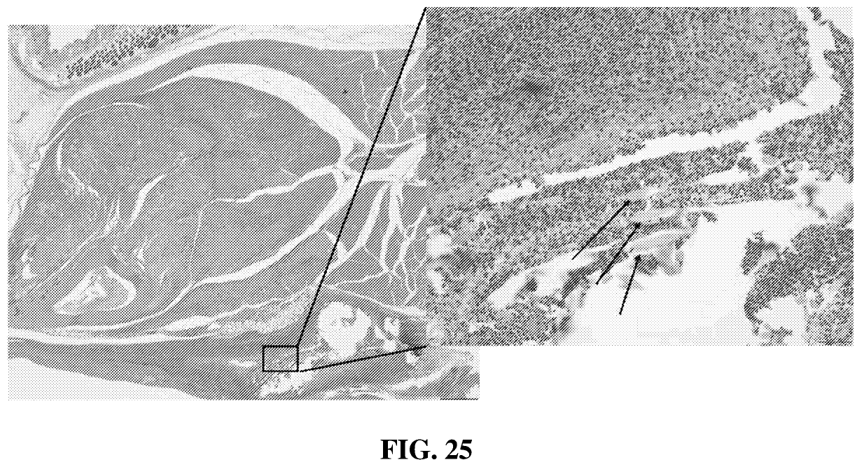

Results & Discussion. Crosslinking GelCad with PEG-SG yields soft hydrogels with loss and storage moduli consistent with other reports for gelatin and collagen-based hydrogels (FIG. 4B). Manipulation of pH was sufficient to alter the crosslinking time without affecting the mechanical properties (FIG. 4A & FIG. 4B). PEG-SG-crosslinked hydrogels excised from the fat pad of BALB/c mice after 72 hours have visual evidence of vessel growth by brightfield microscopy (FIG. 17A and FIG. 17B). Contrast-enhanced ultrasound shows that mTG-crosslinked GelCad, but not GelScram, hydrogels become perfused by host circulation within 1-week (FIG. 18). In the femoral artery ligation model, PEG-SG-crosslinked GelCad hydrogels restored perfusion to the ischemic hindlimb faster than GelScram hydrogels (FIG. 23). PEG-SG-crosslinked GelCad but not GelScram hydrogels prevented hindlimb necrosis and preserved ambulatory function (FIG. 19, FIG. 20, FIG. 21, and FIG. 22). Histology revealed minimal pathology in the hindlimbs of mice that received PEG-SG-crosslinked GelCad hydrogels (FIG. 24), whereas mice that received GelScram hydrogels had extensive necrosis and degeneration across the muscle, bone, and bone marrow (FIG. 25).

### Example 5. GelCad Hydrogels Promote Growth of Smooth Muscle Cells

To better understand how the hydrogels promote arteriogenesis, we embedded primary cortical brain tissue from C57BL6 mice into gelatin, GelScram, and GelCad hydrogels. Samples were imaged after 24 hours, 3 days, and 7 days, upon labeling with fluorescently conjugated antibodies or dyes, to assess vascular growth. In gelatin and GelScram hydrogels, lectin+ vessels could be detected, but they exhibited minimal coverage of α-SMA+ smooth muscle cells. GelCad hydrogels exhibited extensive α-SMA+ smooth muscle cell coverage after 24 hours, and by 7 days, the smooth muscle cells had assembled into mature, ring-like structures around the outer edge of the vessels. Ki-67 labeling showed that in GelCad hydrogels, many cell types in and around the vessels are proliferating. These data show that inclusion of the cadherin peptide on the hydrogels activates smooth muscle cell growth and contributes to the formation of arteriole structures.

### Example 6. GelMA-Cad Hydrogels Support Vascular Growth in Primary Brain Tissue

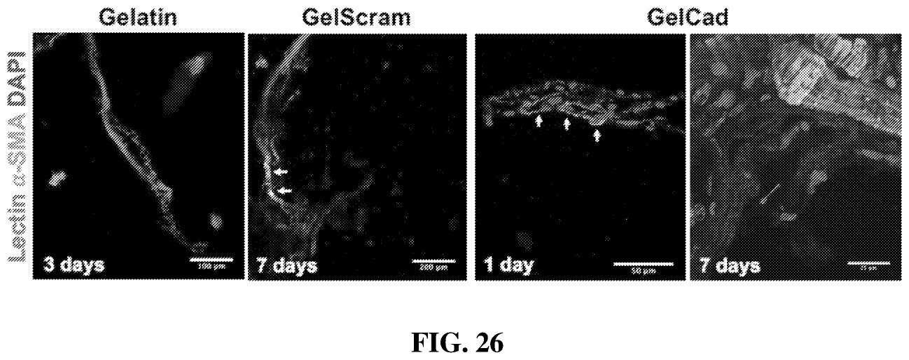

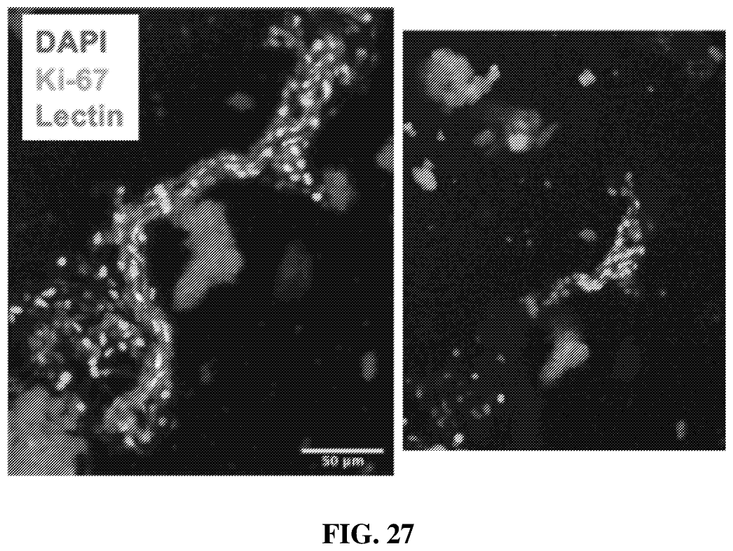

To determine whether hydrogels support vascular growth in primary brain tissue, mouse hippocampus and cortex were dissected and embedded in GelMA-Cad, Matrigel, or Matrigel with vascular endothelial growth factor (VEGF). Brightfield images show that new vessels only sprout in GelMA-Cad hydrogels and not Matrigel hydrogels (FIG. 26 & FIG. 27). Sprouted vessels include arteries and capillaries including of a single lumen. Arteries are larger vessels with multiple claudin-5-positive endothelial cells lined by smooth muscle actin (α-SMA)-positive smooth muscle. Capillaries are smaller vessels with occludin-positive endothelial cells lined with a single layer of neuron-glial antigen 2 (NG2)-positive pericytes. These data show that GelMA-Cad hydrogels support vascular growth in primary brain tissue, whereas Matrigel hydrogels do not, even when provided with a vascular growth factor.

### Example 7. Preparation and Use of Redox-Crosslinking Hydrogel

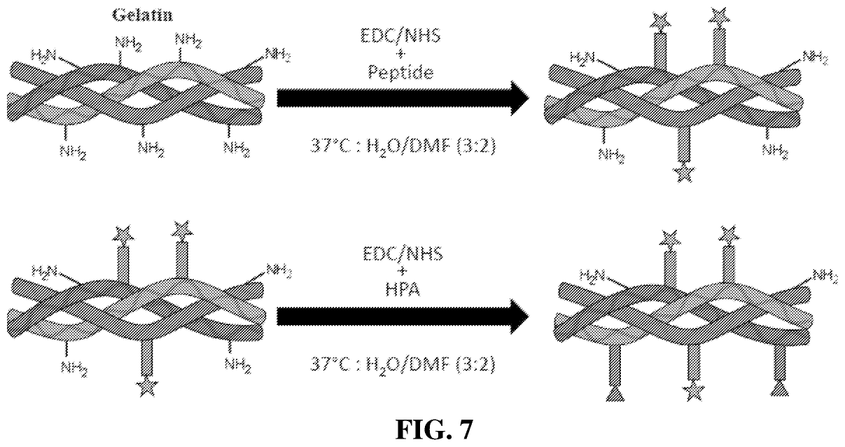

The following process was carried out for conjugating peptide to the gelatin backbone and crosslinking the hydrogel using redox activated crosslinker (redox gel, FIG. 7). Gelatin was reconstituted to a 4-10% solution in PBS. The solution is stirred at 50° C. until dissolved. To activate the peptide solution, EDC (between 5-25 mM concentration), NHS (between 5-25 mM concentration) was dissolved in PBS and DMF (3:2, respectively). The pH was adjusted to 5 and the peptide was added to the solution (between 10 mg to 100 mg), and was allowed to mix for 3 hours. To activate the HPA solution, EDC (between 5-25 mM concentration), NHS (between 5-25 mM concentration) was dissolved in PBS and DMF (3:2, respectively). The pH was adjusted to 5 and HPA was added to the solution (between 10 mg to 4 g), and the mixture was allowed to mix for 3 hours. After 3 hours of mixing, the peptide solution was added to the dissolved gelatin. The pH is adjusted to 5 and allowed to react for another 3 hours with the gelatin. After the 3 hours of Gelatin/peptide mixing and reaction, the HPA solution was added to the solution and allowed to react overnight. The next day the solution was filtered and dialyzed using a tangential flow filtration system (2 kDa pore size). The resulting gel may be crosslinked by known methods using HRP and H₂O₂.

¹H-NMR spectrum showed the successful preparation of the resulting redox gel (Gelatin-Cad-HPA). The presence of HPA and Cad peptide structures are confirmed by ¹H-NMR signals (˜1.10 and 2.50-3.10 ppm).

### Example 8. GelCad Hydrogels Support Vascular Growth from Primary Human Brain Tissue

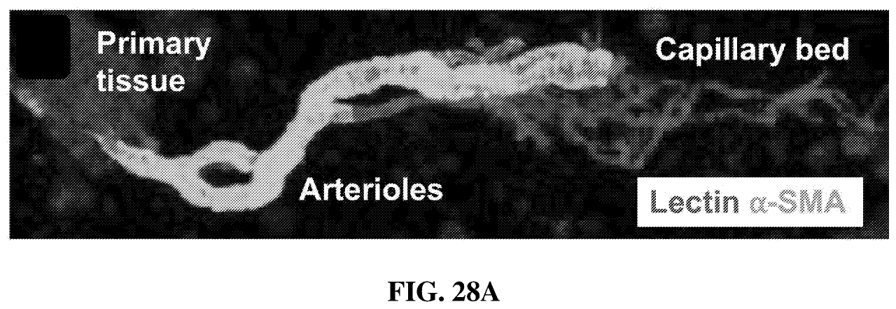

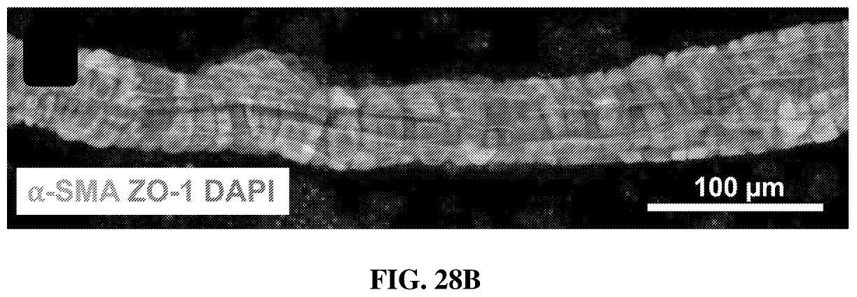

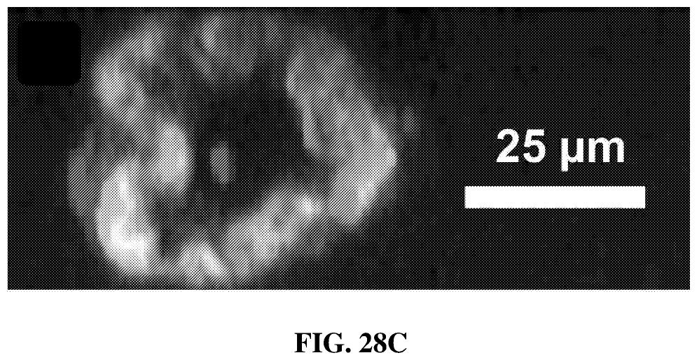

To determine whether redox-crosslinking hydrogels support vascular outgrowth in primary human brain tissue, cortex was dissected and embedded in a redox-crosslinking hydrogel. Fluorescent images show that new vessels sprout in primary human brain tissue when embedded in redox-crosslinking hydrogels. Vascular outgrowth begins 24 hours after embedding in the hydrogel. Vascular outgrowth continues throughout 48 hours and 4 days after embedding the brain tissue in the hydrogel. These data show that redox-crosslinking hydrogels support vascular sprouting in primary human brain tissue. This can be seen in FIG. 28A, FIG. 28B, and FIG. 28C.

It is understood that the foregoing detailed description and accompanying examples are merely illustrative and are not to be taken as limitations upon the scope of the invention, which is defined solely by the following claims.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A method of promoting arteriogenesis for treating a disorder in a subject, the method comprising administering to an area of the subject in need thereof a therapeutically effective amount of a biomaterial, the biomaterial comprising a crosslinked hydrogel, and a peptide chemically attached to the hydrogel and comprising an extracellular epitope of a cadherin protein, wherein the biomaterial provides artery growth, arteriole growth, or a combination thereof in the area of administration.

Clause 2. The method of clause 1, wherein the artery growth, arteriole growth, or a combination thereof occurs within about 1 day to about 14 days.

Clause 3. The method of clause 1 or clause 2, wherein the artery growth, arteriole growth, or a combination thereof corresponds to providing a vessel having a diameter of about 0.1 mm to about 1 mm.

Clause 4. The method of any one of clauses 1-3, wherein the biomaterial increases blood flow in the area of administration compared to blood flow in the area prior to administration as measured by perfusion index.

Clause 5. The method of any one of clauses 1-4, wherein the area of administration is an area of the subject having impaired blood flow.

Clause 6. The method of any one of clauses 1-5, wherein the administration of the biomaterial repairs damage caused by ischemia in said subject as measured by modified ischemia score.

Clause 7. The method of any one of clauses 1-6, wherein the disorder is selected from the group consisting of ischemic heart disease, coronary artery disease, ischemic heart failure, peripheral artery disease, critical limb ischemia, diabetic foot ulcer, organ transplant, skin wound, and bone fracture.

Clause 8. The method of any one of clauses 1-7, wherein the subject is human.

Clause 9. The method of any one of clauses 1-8, wherein the hydrogel comprises a polymeric material at about 1% to about 20% by weight of the hydrogel.

Clause 10. The method of clause 9, wherein the polymeric material comprises gelatin.

Clause 11. The method of any one of clauses 1-10, wherein the peptide comprises SEQ ID NO: 1, SEQ ID NO: 2, a variant of SEQ ID NO: 1 or SEQ ID NO: 2, or a combination thereof.

Clause 12. The method of any one of clauses 1-11, wherein the hydrogel is crosslinked via a crosslinker comprising an acrylate group, a phenol group, a multivalent polyethylene glycol (PEG), or a combination thereof, or via non-covalent interactions.

Clause 13. The method of any one of clauses 1-12, wherein the biomaterial has a stiffness of about 800 Pa to about 5 kPa.

Clause 14. The method of any one of clauses 1-13, wherein the biomaterial comprises a plurality of pores, each individual pore having a diameter of about 20 μm to about 80 μm.

Clause 15. The method of any one of clauses 1-14, wherein the biomaterial further comprises a plurality of cells, a growth factor, or a combination thereof.

Clause 16. The method of any one of clauses 1-15, wherein the biomaterial is administered without any cells.

Clause 17. A biomaterial comprising a crosslinked hydrogel comprising a multivalent polyethylene glycol (PEG) crosslinker; and a peptide chemically attached to the hydrogel and comprising an extracellular epitope of a cadherin protein.

Clause 18. The biomaterial of clause 17, wherein the hydrogel comprises a polymeric material at about 1% to about 20% by weight of the hydrogel.

Clause 19. The biomaterial of clause 18, wherein the polymeric material comprises gelatin.

Clause 20. The biomaterial of clause 19, wherein the hydrogel comprises the peptide at a degree of functionalization of about 10% to about 70% as measured by the amount of valine of the peptide relative to the amount of phenylalanine of the hydrogel per nuclear magnetic resonance.

Clause 21. The biomaterial of any one of clauses 17-20, wherein the multivalent PEG crosslinker is a multivalent PEG succinimidyl glutarate.

Clause 22. The biomaterial of any one of clauses 17-21, wherein the multivalent PEG crosslinker has a number average molecular weight of about 1 kDa to about 40 kDa.

Clause 23. The biomaterial of any one of clauses 17-22, wherein the peptide comprises SEQ ID NO: 1, SEQ ID NO: 2, a variant of SEQ ID NO: 1 or SEQ ID NO: 2, or a combination thereof.

Clause 24. The biomaterial of any one of clauses 17-23, having a stiffness of about 800 Pa to about 5 kPa.

Clause 25. The biomaterial of any one of clauses 17-24, comprising a plurality of pores, each individual pore having a diameter of about 20 μm to about 80 μm.

Clause 26. A biomaterial comprising a crosslinked hydrogel; and a peptide chemically attached to the hydrogel and comprising an extracellular epitope of a cadherin protein.

Clause 27. The biomaterial of clause 26, wherein the hydrogel comprises a polymeric material at about 1% to about 20% by weight of the hydrogel.

Clause 28. The biomaterial of clause 27, wherein the polymeric material comprises gelatin.

Clause 29. The biomaterial of any one of clauses 26-28, wherein the peptide comprises SEQ ID NO: 1, SEQ ID NO. 2, a variant of SEQ ID NO: 1 or SEQ ID NO: 2, or a combination thereof.

Clause 30. The biomaterial of any one of clauses 28-29, wherein the hydrogel comprises the peptide at a degree of functionalization of about 10% to about 70% as measured by the amount of valine of the peptide relative to the amount of phenylalanine of the hydrogel per nuclear magnetic resonance.

Clause 31. The biomaterial of any one of clauses 26-30, wherein the hydrogel is crosslinked via a crosslinker comprising an acrylate group, a phenol group, a multivalent polyethylene glycol (PEG), or a combination thereof, or via non-covalent interactions.

Clause 32. The biomaterial of any one of clauses 26-31, wherein the multivalent PEG crosslinker is a multivalent PEG succinimidyl glutarate.

Clause 33. The biomaterial of any one of clauses 26-32, wherein the multivalent PEG has a number average molecular weight of about 1 kDa to about 40 kDa.

Clause 34. The biomaterial of any one of clauses 26-33, wherein the biomaterial has a stiffness of about 800 Pa to about 5 kPa.

Clause 35. The biomaterial of any one of clauses 26-34, wherein the biomaterial comprises a plurality of pores, each individual pore having a diameter of about 20 μm to about 80 μm.

Clause 36. The biomaterial of any one of clauses 26-35, wherein the biomaterial further comprises a plurality of cells, a growth factor, or a combination thereof.

### SEQUENCES

SEQ ID NO: 1

HAVDIGGGC

SEQ ID NO: 2

HAVDIGGGCE

What is claimed is:

1. A method of promoting arteriogenesis for treating a disorder in a subject, the method comprising administering to an area of the subject in need thereof a therapeutically effective amount of a biomaterial, the biomaterial comprising
a crosslinked hydrogel, and
a peptide chemically attached to the hydrogel and comprising an extracellular epitope of a cadherin protein,
wherein the biomaterial provides artery growth, arteriole growth, or a combination thereof in the area of administration.

2. The method of claim 1, wherein the artery growth, arteriole growth, or a combination thereof occurs within about 1 day to about 14 days.

3. The method of claim 1, wherein the artery growth, arteriole growth, or a combination thereof corresponds to providing a vessel having a diameter of about 0.1 mm to about 1 mm.

4. The method of claim 1, wherein the biomaterial increases blood flow in the area of administration compared to blood flow in the area prior to administration as measured by perfusion index.

5. The method of claim 1, wherein the area of administration is an area of the subject having impaired blood flow.

6. The method of claim 1, wherein the administration of the biomaterial repairs damage caused by ischemia in said subject as measured by modified ischemia score.

7. The method of claim 1, wherein the disorder is selected from the group consisting of ischemic heart disease, coronary artery disease, ischemic heart failure, peripheral artery disease, critical limb ischemia, diabetic foot ulcer, organ transplant, skin wound, and bone fracture.

8. The method of claim 1, wherein the subject is human.

9. The method of claim 1, wherein the hydrogel comprises a polymeric material at about 1% to about 20% by weight of the hydrogel.

10. The method of claim 9, wherein the polymeric material comprises gelatin.

11. The method of claim 1, wherein the peptide comprises SEQ ID NO: 1, SEQ ID NO: 2, a variant of SEQ ID NO: 1 or SEQ ID NO: 2, or a combination thereof.

12. The method of claim 1, wherein the hydrogel is crosslinked via a crosslinker comprising an acrylate group, a phenol group, a multivalent polyethylene glycol (PEG), or a combination thereof, or via non-covalent interactions.

13. The method of claim 1, wherein the biomaterial has a stiffness of about 800 Pa to about 5 kPa.

14. The method of claim 1, wherein the biomaterial comprises a plurality of pores, each individual pore having a diameter of about 20 μm to about 80 μm.

15. The method of claim 1, wherein the biomaterial further comprises a plurality of cells, a growth factor, or a combination thereof.

16. The method of claim 1, wherein the biomaterial is administered without any cells.

17. A biomaterial comprising:
a crosslinked hydrogel comprising a multivalent polyethylene glycol (PEG) crosslinker; and
a peptide chemically attached to the hydrogel and comprising an extracellular epitope of a cadherin protein.

18. The biomaterial of claim 17, wherein the hydrogel comprises a polymeric material at about 1% to about 20% by weight of the hydrogel.

19. The biomaterial of claim 18, wherein the polymeric material comprises gelatin.

20. The biomaterial of claim 19, wherein the hydrogel comprises the peptide at a degree of functionalization of about 10% to about 70% as measured by the amount of valine of the peptide relative to the amount of phenylalanine of the hydrogel per nuclear magnetic resonance.

21. The biomaterial of claim 17, wherein the multivalent PEG crosslinker is a multivalent PEG succinimidyl glutarate.

22. The biomaterial of claim 17, wherein the multivalent PEG crosslinker has a number average molecular weight of about 1 kDa to about 40 kDa.

23. The biomaterial of claim 17, wherein the peptide comprises SEQ ID NO: 1, SEQ ID NO:
2, a variant of SEQ ID NO: 1 or SEQ ID NO: 2, or a combination thereof.

24. The biomaterial of claim 17, having a stiffness of about 800 Pa to about 5 kPa.

25. The biomaterial of claim 17, comprising a plurality of pores, each individual pore having a diameter of about 20 μm to about 80 μm.


SEQUENCE LISTING (2 sequences)

SEQ ID NO: 1 - protein, 9 aa, Artificial Sequence
  Synthetic
HAVDIGGGC

SEQ ID NO: 2 - protein, 10 aa, Artificial Sequence
  Synthetic
HAVDIGGGCE